(19) United States
(12) Patent Application Publication
(10) Pub. No.: US 2014/0162955 A1
(43) Pub. Date: Jun. 12, 2014

(54) Antagonizing Heparin With Salicylamide Compounds And Histamine Blocking Agents

(71) Applicants: Richard W. Scott, West Chester, PA (US); Bozena Korczak, Wayne, PA (US); Nicholas Landekic, Radnor, PA (US)

(72) Inventors: Richard W. Scott, West Chester, PA (US); Bozena Korczak, Wayne, PA (US); Nicholas Landekic, Radnor, PA (US)

(73) Assignee: CELLCEUTIX CORPORATION, Beverly, MA (US)

(21) Appl. No.: 14/100,811

(22) Filed: Dec. 9, 2013

Related U.S. Application Data
(60) Provisional application No. 61/735,096, filed on Dec. 10, 2012.

(51) Int. Cl. C07C 237/44; C07C 279/14; C07K 5/06; A61K 38/05; A61K 31/495; A61K 31/4164; A61K 31/451; A61K 31/445; A61K 31/4402; A61K 31/5415; A61K 31/167; A61K 31/135

(52) U.S. Cl. CPC C07C 237/44; A61K 31/167; C07C 279/14; C07K 5/06; A61K 38/05; A61K 31/135; A61K 31/4164; A61K 31/451; A61K 31/445; A61K 31/4402; A61K 31/5415; A61K 31/495
USPC 514/14.9

(57) ABSTRACT

The present disclosure provides combinations of salicylamide compounds, or pharmaceutically acceptable salts thereof, and histamine blocking agents, or pharmaceutically acceptable salts thereof, for antagonizing unfractionated heparin, low molecular weight heparin, or a heparin/low molecular weight heparin derivative.

### FIELD

The present disclosure is directed, in part, to antagonizing unfractionated heparin, low molecular weight heparin, or a heparin/low molecular weight heparin derivative by administering a salicylamide compound, or pharmaceutically acceptable salt thereof, and a histamine blocking agent, or a pharmaceutically acceptable salt thereof, and pharmaceutical compositions therefor.

### BACKGROUND

Treatment and prevention of thrombosis are major clinical issues for medical and surgical patients. Heparin, a highly sulfated polysaccharide, is commonly used as prophylaxis against venous thromboembolism and to treat venous thrombosis, pulmonary embolism, unstable angina and myocardial infarction (see, for example, Walenga et al., “Factor Xa inhibition in mediating antithrombotic actions: application of a synthetic heparin pentasaccharide” In. Paris: Universite Pierre et Marie Curie, Paris VI; 1987; and Hirsh et. al., Chest, 2001, 119, 64-94). Heparin is also used as an anticoagulant during the extracorporeal blood circulation for kidney dialysis and coronary bypass surgery. Although heparin is an efficacious anticoagulant, there are many limitations associated with its clinical use. For example, heparin's heterogeneity and polydispersity lead to nonspecific protein binding and poorly predictive pharmacokinetic properties upon subcutaneous (s.c.), and even intravenous, injection (see, for example, Bendetowicz et. al., Thromb. Hemostasis., 1994, 71, 305-313). As a result, infusions of unfractionated heparin (UFH) are performed in the hospital where its anticoagulant effect can be measured to minimize the risk of bleeding. In addition to hemorrhage, administration of UFH is associated with 1-2% incidence of heparin-induced thrombocytopenia (HIT) (see, for example, Morabia, Lancet, 1986, 1, 1278-1279; Mureebe et. al., Vasc. Endovasc. Surg., 2002, 36, 163-170; and Lubenow et. al., Chest, 2002, 122, 37-42).

To address some of the shortcomings of UFH, low molecular weight heparins (LMWHs) have been developed. LMWHs are fragments of UFH produced by chemical or enzymatic depolymerization (see, for example, Hirsh et. al., Blood, 1992, 79, 1-17). Due to their smaller size and lower polydispersity, LMWHs are more reproducibly bioavailable after subcutaneous administration and have more predictable pharmacokinetics leading to greater safety (see, for example, Ofosu et. al., “Mechanisms of action of low molecular weight heparins and heparinoids.” In: Hirsh J (ed). Antithrombotic Therapy, Bailliere's Clinical Haematology (Volume 3). London, UK: Bailliere Tindall, 1990, pp. 505-529). The smaller size of LMWHs is also associated with a lower ratio of anti-thrombin to anti-FXa activity (see, for example, Hirsh et. al., Chest, 2001, 119, 64-94). LMWHs are being used with greater frequency owing to their ease of administration, longer duration or action and reduced incidence of heparin-induced thrombocytopenia (see, for example, Hirsh et. al., Chest, 2004, 126 (Suppl 3), 188S-203S). LMWHs are commonly used to treat deep vein thrombosis, unstable angina, and acute pulmonary embolism, as well as thromboprophylactic agents in a wide range of clinical situations including orthopedic surgery, high risk pregnancy, and cancer therapy (see, for example, Hirsh et. al., Chest, 2004, 126 (Suppl 3), 188S-203S; Becker, J. Thrombosis and Thrombolysis, 1999, 7, 195; Antman et. al., Circulation, 1999, 100, 1593-601; Cohen et. al., New England J. Med., 1997, 337, 447; and Lee et. al., J Clin. Oncol., 2005, 23, 2123-9).

Fondaparinux is a heparin-derived pentasaccharide that represents the smallest fragment of heparin that is capable of accelerating antithrombin-mediated factor Xa inhibition (see, for example, Walenga et. al., Exp. Opin. Invest. Drugs, 2005, 14, 847-58). Fondaparinux is currently approved for the prophylaxis of deep vein thrombosis following hip repair and/or replacement, knee replacement and abdominal surgery and the treatment of DVT/PE when used in conjunction with warfarin. The most common complication of anticoagulation with LMWHs is hemorrhage. Many published clinical studies report 1% to 4% major (life-threatening) bleeding associated with LMWH therapy and there is a 5-fold increase in the overall death rate for acute coronary syndrome patients receiving anticoagulant therapy that experience major bleeding (see, for example, Hirsh et. al., Chest, 2001, 119, 64-94; and Mehta et. al., J. Am. Coll. Cardiol., 2007, 50, 1742-1751).

Protamine, an arginine-rich heterogeneous peptide mixture isolated from fish sperm, is used routinely to neutralize the effects of heparin in patients who bleed while under treatment (see, for example, Ando et. al., in Kleinzeller, A. (ed): “Protamine: Molecular biology, biochemistry and biophysics” Vol 12, 1973, New York, Springer-Verlag, 1-109). Polycationic protamine binds to anionic heparin through electrostatic interactions, thereby neutralizing the anticoagulant effects of heparin. Although protamine is commonly used to neutralize UFH following coronary bypass surgery, it is unable to completely reverse the anticoagulant effects of LMWHs (see, for example, Hubbard et. al., Thromb. Haemost., 1985, 53, 86-89; Poon et. al., Thromb. Haemost., 1982, 47, 162-165; Massonnet-Castel et. al., Haemostasis, 1986, 16, 139-146; and Doutremepuich et. al., Semin. Thromb. Hemost., 1985, 11, 318-322) or fondaparinux (see, for example, Walenga, “Factor Xa inhibition in mediating antithrombotic actions: application of a synthetic heparin pentasaccharide” In. Paris: Universite Pierre et Marie Curie, Paris VI; 1987).

In addition, use of protamine for heparin reversal is associated with adverse reactions including systemic vasodilation and hypotension, bradycardia, pulmonary artery hypertension, pulmonary vasoconstriction, thrombocytopenia, and neutropenia (see, for example, Metz et. al., “Protamine and newer heparin antagonists” in Stoetling, R. K. (ed): Pharmacology and Physiology in Anesthetic Practice. Vol. 1. Philadelphia, Pa., J B Lippincott, 1-15, 1994; Weiler et. al., J. Allergy Clin. Immunol., 1985, 75, 297-303; Horrow, Anest. Analg., 1985, 64, 348-361; and Porsche et. al., Heart Lung J. Acute Crit. Care, 1999, 28, 418-428).

Therefore, there is a strong medical need for the development of a safe and effective antagonist for UFH and/or LMWH. The lack of an effective antagonist has limited the clinical use of the LMWHs and fondaparinux, especially in bypass procedures and instances where near term surgical procedures may be needed. There is also a strong medical need for an efficacious, nontoxic substitute for protamine. Further, efficacy against the anticoagulation properties of the LMWHs would substantially address an important and expanding medical market for which no effective antidote is available.

### SUMMARY

The present disclosure provides pharmaceutical compositions comprising: a) a salicylamide compound of Formula I:

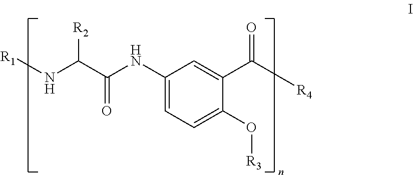

wherein: n is 2 to 10; R₁ is H or

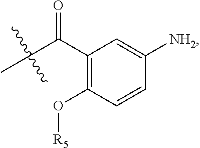

where R₅ is H or C₁ to C₉ straight or branched alkyl optionally substituted with one or more —NH₂, —N(CH₃)₂, or

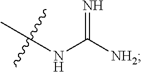 [SMILES: *NC(=N)N]

each R₂ is, independently, C₁ to C₉ straight or branched alkyl optionally substituted with one or more —NH₂, —N(CH₃)₂, or

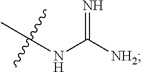 [SMILES: *NC(=N)N]

each R₃ is, independently, C₁ to C₉ straight or branched alkyl optionally substituted with one or more —NH₂, —N(CH₃)₂, or

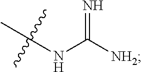 [SMILES: *NC(=N)N]

### and R₄ is OH, NH₂, or

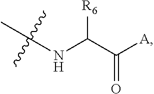

where A is OH or NH₂, and R₆ is H or C₁ to C₉ straight or branched alkyl optionally substituted with one or more —NH₂, —N(CH₃)₂, or

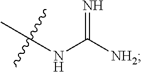 [SMILES: *NC(=N)N]

or a pharmaceutically acceptable salt thereof; and b) one or more histamine blocking agents, or a pharmaceutically acceptable salt thereof.

The present disclosure also provides methods of antagonizing unfractionated heparin, low molecular weight heparin, or a heparin/low molecular weight heparin derivative in a mammal comprising administering a pharmaceutical composition described herein to the mammal.

The present disclosure also provides methods of antagonizing unfractionated heparin, low molecular weight heparin, or a heparin/low molecular weight heparin derivative in a mammal comprising: administering a histamine blocking agent to the mammal; and administering a salicylamide compound to the mammal.

The present disclosure also provides pharmaceutical compositions described herein for antagonizing unfractionated heparin, low molecular weight heparin, or a heparin/low molecular weight heparin derivative.

The present disclosure also provides pharmaceutical compositions described herein for use in the manufacture of a medicament for antagonizing unfractionated heparin, low molecular weight heparin, or a heparin/low molecular weight heparin derivative.

The present disclosure also provides uses of pharmaceutical compositions described herein for antagonizing unfractionated heparin, low molecular weight heparin, or a heparin/low molecular weight heparin derivative.

The present disclosure also provides uses of pharmaceutical compositions described herein for the manufacture of a medicament for antagonizing unfractionated heparin, low molecular weight heparin, or a heparin/low molecular weight heparin derivative.

### BRIEF DESCRIPTION OF THE DRAWINGS

FIG. 1 shows representative histamine release from RBL cells pre-treated with heparin or enoxaparin and Compound 100.

FIGS. 2A-D show representative Mean Arterial Pressures for Compound 100+diphenhydramine (DPH) (2A), Compound 100+cimetidine (CIM) (2B), Compound 100+NO synthase inhibitor (L-NAME) (2c), and Compound 100+diphenhydramine/cimetidine (DPH/CIM) (2D).

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, decyl, 4,4-dimethylpentyl, 2,2,4-trimethylpentyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “amino” means —NH₂.

As used herein, the term “antagonize” or “antagonizing” means reducing or completely eliminating an effect, such as the anticoagulant effect of heparin.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a heparin or LMWH with a compound includes the administration of a compound to an individual or patient, such as a human, having been administered a heparin, as well as, for example, introducing a compound into a sample containing a cellular or purified preparation containing the heparin, or before an individual has been administered a heparin.

As used herein, the term “guanidino” means —NH(═NH)NH₂.

As used herein, the term “heparin” means naturally occurring unfractionated heparin and low molecular weight heparin, which can be used as an anticoagulant in diseases that feature thrombosis, as well as for prophylaxis in situations that lead to a high risk of thrombosis. The term “heparin” further includes anticoagulant agents that are derivatives of unfractionated heparin and/or LMWH, for example, by chemical modification, through enzymatic process, or direct synthesis. Examples of such heparin derivatives (for example, chemically modified unfractionated heparin and/or LMWH; or pentasaccharide) include fondaparinux. Examples of LMWH include, but are limited to, enoxaparin, reviparin, and tinzaparin.

As used herein, the term “hydroxy” or “hydroxyl” means an —OH group.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. In some embodiments, the animal or mammal will be in need of antagonizing a heparin, a low molecular weight heparin, or a heparin/low molecular weight heparin derivative while reducing or minimizing unwanted skin or other tissue reactions to a salicylamide compound.

As used herein, the phrase “from 1 to 5” means 1, 2, 3, 4, or 5.

As used herein, the term “isolated” means that the compounds described herein are separated from other components of either (a) a natural source, such as a plant or cell, such as a bacterial culture, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present disclosure also includes quaternary ammonium salts of the compounds described herein, where the compounds have one or more tertiary amine moiety.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the phrase “substantially isolated” means a compound that is at least partially or substantially separated from the environment in which it is formed or detected.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₆cycloalkyl, C₅-C₆aryloxy, —CN, —OH, oxo, halo, haloalkyl, —NO₂, —CO₂H, —NH₂, —NH(C₁-C₈alkyl), —N(C₁-C₈alkyl)₂, —NH(C₆aryl), —N(C₅-C₆aryl)₂, —CHO, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes of this disclosure, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment.

At various places in the present specification, substituents of compounds may be disclosed in groups or in ranges. It is specifically intended that the disclosure include each and every individual subcombination of the members of such groups and ranges. For example, the term “C₁ to C₆ alkyl” is specifically intended to individually disclose methyl, ethyl, propyl, C₄alkyl, C₅alkyl, and C₆alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

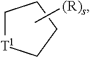

then it is understood that substituent R can occur s number of times on the ring, and R can be a different moiety at each occurrence. Further, in the above example, where the variable T¹ is defined to include hydrogens, such as when T¹ is CH₂, NH, etc., any H can be replaced with a substituent.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

It is understood that the present disclosure encompasses the use, where applicable, of stereoisomers, diastereomers and optical stereoisomers of the compounds of the disclosure, as well as mixtures thereof. Additionally, it is understood that stereoisomers, diastereomers, and optical stereoisomers of the compounds of the disclosure, and mixtures thereof, are within the scope of the disclosure. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the disclosure unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds are also included within the scope of the disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

Compounds can also include various charged states. For example, one or more moieties of any of the compounds described herein can be charged. In some instances, any moiety having an amino group can be —NH₃⁺. Thus, each amino group existing in any compound described herein can, independently, be either —NH₂ or —NH₃⁺.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs.

Some of the compounds may be capable of adopting amphiphilic conformations that allow for the segregation of polar and nonpolar regions of the molecule into different spatial regions and provide the basis for a number of uses. For example, some compounds may adopt amphiphilic conformations that are capable of binding to heparin (including, for example, unfractionated heparin, low molecular weight heparin, and synthetically modified heparin or low molecular heparin derivatives). Although not wishing to be bound by any particular theory, it is believed that compounds can interact with heparin through electrostatic interactions.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

The present disclosure provides pharmaceutical compositions comprising:

a) a salicylamide compound of Formula I:

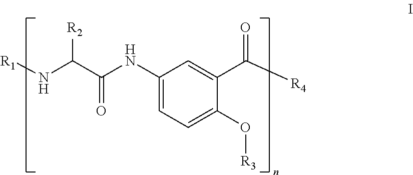

wherein:

n is 2 to 10;

R₁ is H or

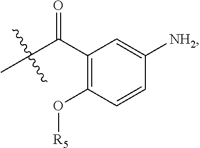

where R₅ is H or C₁ to C₉ straight or branched alkyl optionally substituted with one or more —NH₂, —N(CH₃)₂, or

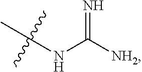 [SMILES: *NC(=N)N]

or another suitable substituent;

each R₂ is, independently, C₁ to C₉ straight or branched alkyl optionally substituted with one or more —NH₂, —N(CH₃)₂, or

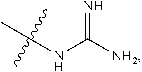 [SMILES: *NC(=N)N]

or another suitable substituent;

each R₃ is, independently, C₁ to C₉ straight or branched alkyl optionally substituted with one or more —NH₂, —N(CH₃)₂, or

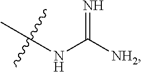 [SMILES: *NC(=N)N]

or another suitable substituent; and

R₄ is OH, NH₂, or

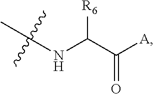

where A is OH or NH₂, and R₆ is H or C₁ to C₉ straight or branched alkyl optionally substituted with one or more —NH₂, —N(CH₃)₂, or

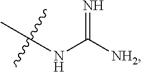 [SMILES: *NC(=N)N]

or another suitable substituent;
or a pharmaceutically acceptable salt thereof; and

b) one or more histamine blocking agents, or a pharmaceutically acceptable salt thereof.

In some embodiments, n is 3 to 8. In some embodiments, n is 3 to 5. In some embodiments, n is 3 or 4.

In some embodiments, R₁ is H.

In some embodiments, each R₂ is, independently, C₃ to C₅ straight or branched alkyl optionally substituted with one or more —NH₂ or

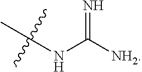 [SMILES: *NC(=N)N]

In some embodiments, each R₂ is, independently, C₃ or C₄ straight alkyl optionally substituted with one —NH₂ or

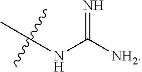 [SMILES: *NC(=N)N]

In some embodiments, each R₂ is, independently, C₃ or C₄ straight alkyl substituted with one —NH₂ or

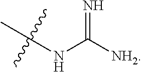 [SMILES: *NC(=N)N]

In some embodiments, each R₃ is, independently, C₁ to C₉ straight or branched alkyl. In some embodiments, each R₃ is, independently, C₁ to C₃ straight alkyl.

In some embodiments, R₄ is OH, NH₂, or

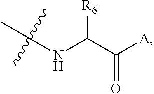

where A is NH₂, and R₆ is C₁ to C₉ straight or branched alkyl optionally substituted with one —NH₂, —N(CH₃)₂, or

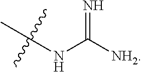 [SMILES: *NC(=N)N]

In some embodiments, R₄ is OH or NH₂.

In some embodiments, n is 3 to 5; R₁ is H; each R₂ is, independently, C₃ to C₅ straight alkyl optionally substituted with one —NH₂, —N(CH₃)₂, or

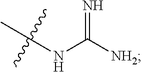 [SMILES: *NC(=N)N]

each R₃ is, independently, C₁ to C₃ straight alkyl optionally substituted with one —NH₂; and R₄ is OH or NH₂.

In some embodiments, n is 3 or 4; R₁ is H; each R₂ is, independently, C₃ or C₄ straight alkyl substituted with one —NH₂ or

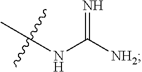 [SMILES: *NC(=N)N]

each R₃ is, independently, C₁ or C₂ alkyl; and R₄ is NH₂.

In some embodiments, the salicylamide compound is chosen from:

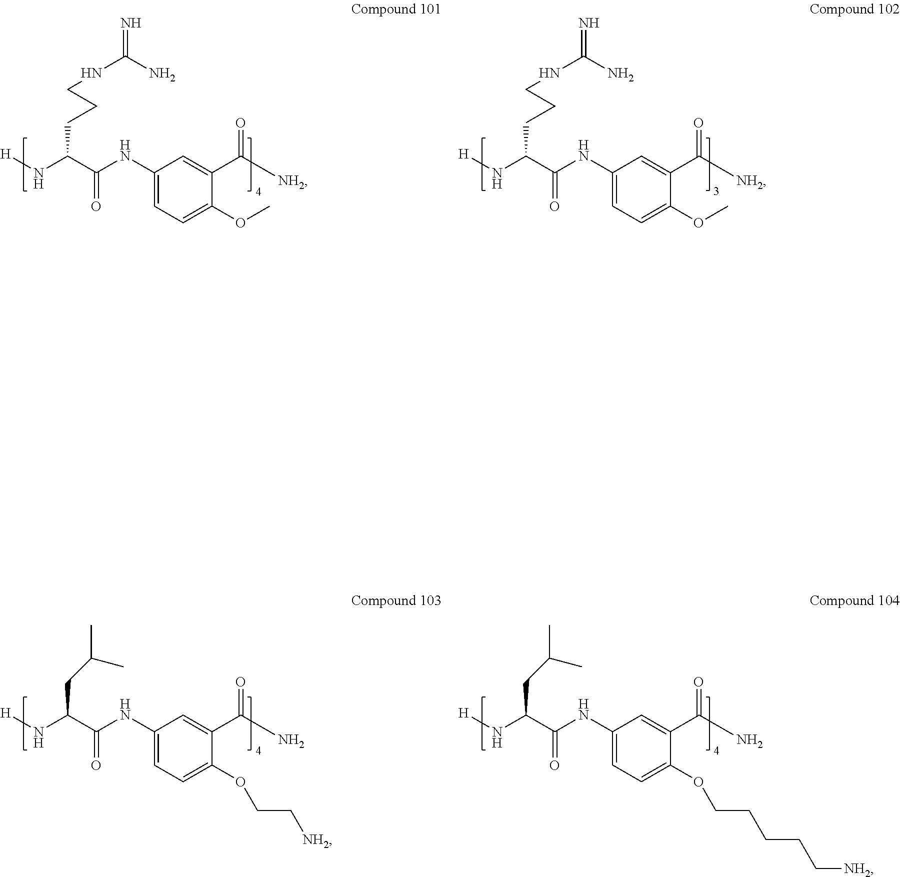

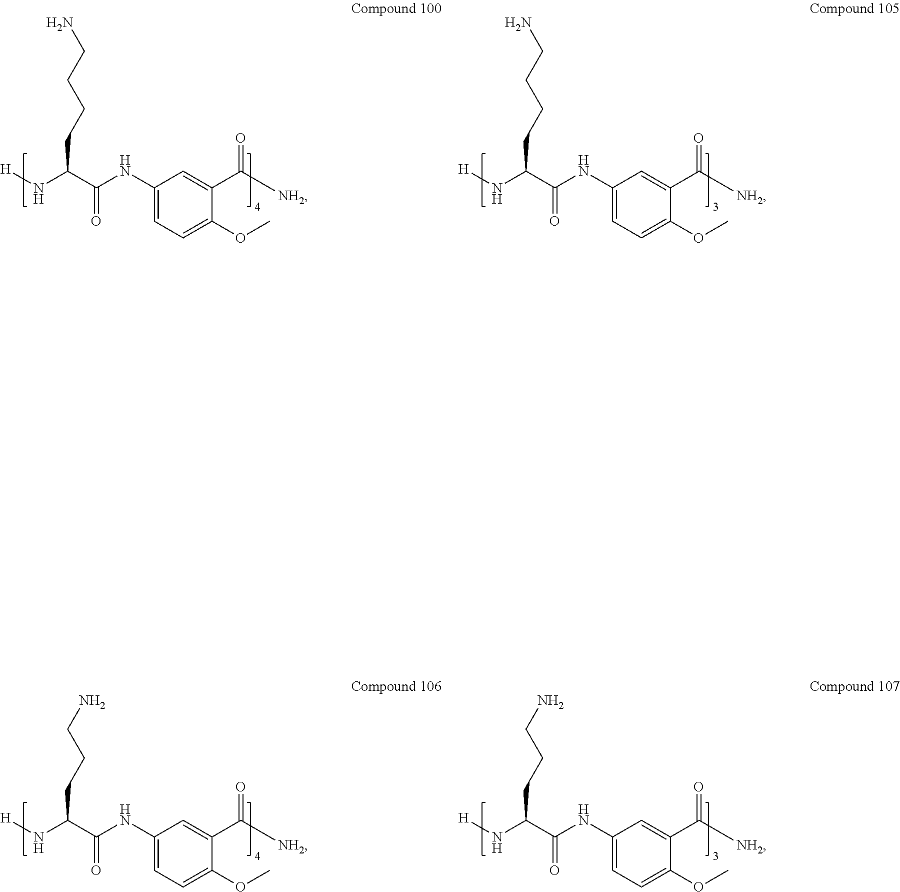

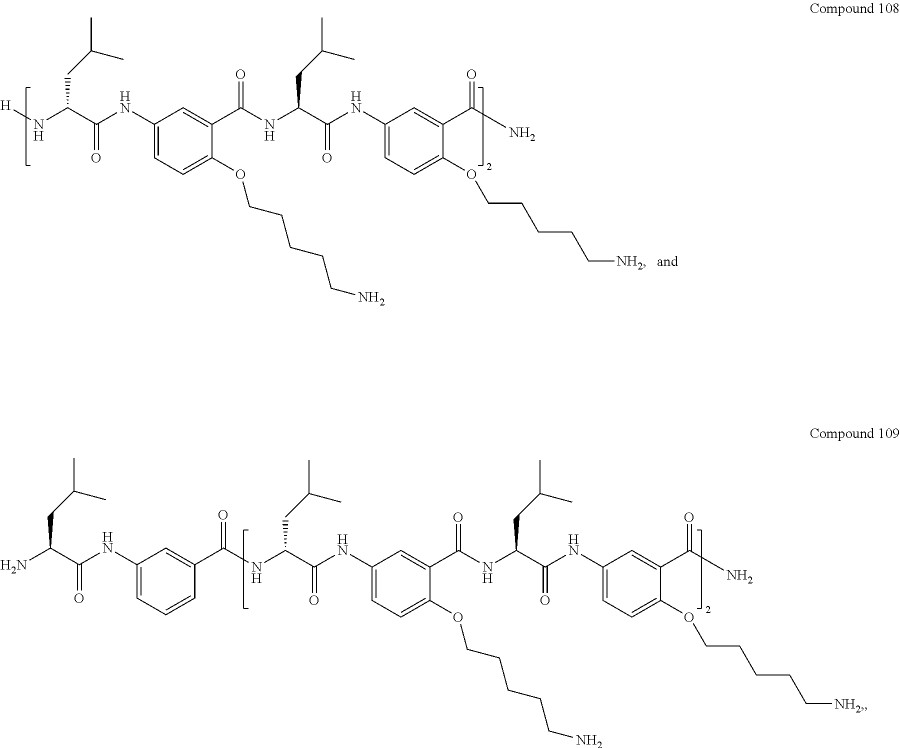

or a pharmaceutically acceptable salt thereof. In some embodiments, the salicylamide compound is

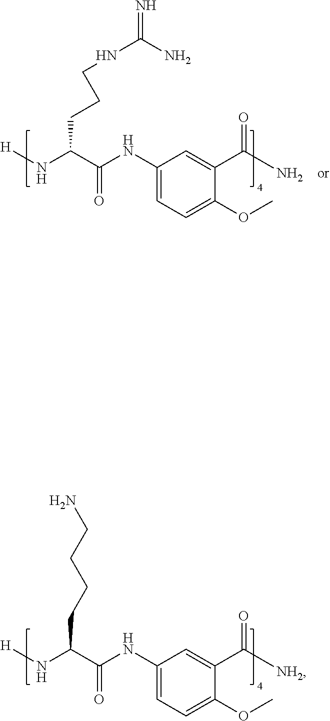

or a pharmaceutically acceptable salt thereof.

In some embodiments, the histamine blocking agent can be an H1-receptor and/or H2-receptor antagonist or is chosen from diphenhydramine (Benadryl), loratadine (Claritin), fexofenadine (Allegra), chlorpheniramine (Chlor-Tripalon), cimetidine (Tagamet), brompheniramine (Dimetane), dimenhydrinate (Gravol), promethazine (Phenergan), hydroxyzine (Atarax), cyproheptadine (Periactin), azatadine (Zadine), and cetirizine (Reactine), or a pharmaceutically acceptable salt thereof. In some embodiments, the histamine blocking agent is diphenhydramine. In some embodiments, a combination of two or more histamine blocking agents is used. In some embodiments, the combination is diphenhydramine and cimetidine.

In some embodiments, the salicylamide compound is

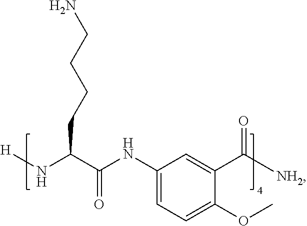

or a pharmaceutically acceptable salt thereof, and the histamine blocking agent is diphenhydramine, cimetidine, or a combination of diphenhydramine and cimetidine.

The syntheses of compounds described herein can be carried out by routine and/or known methods such as those disclosed in, for example, WO 11/50162, which is incorporated herein by reference in its entirety. Numerous pathways are available to incorporate polar and nonpolar side chains. Phenolic groups on the monomer can be alkylated. Alkylation of the commercially available phenol will be accomplished with standard Williamson ether synthesis for the non-polar side chain with ethyl bromide as the alkylating agent. Polar sidechains can be introduced with bifunctional alkylating agents such as BOC-NH(CH₂)₂Br. Alternately, the phenol group can be alkylated to install the desired polar side chain function by employing the Mitsonobu reaction with BOC-NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternately, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

Compounds described herein can also be synthesized by solid-phase synthetic procedures well know to those of skill in the art (see, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dorwald, F. Z., Organic Synthesis on Solid Phase: Supports, Linkers, Reactions, 2nd Ed., Wiley-VCH, Weinheim (2002)).

The compounds described herein can also be designed using computer-aided computational techniques, such as de novo design techniques, to embody the amphiphilic properties. In general, de novo design of compounds is performed by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds can be synthesized from this selected library to verify structures and test their biological activity. Novel molecular dynamic and coarse grain modeling programs have also been developed for this approach because existing force fields developed for biological molecules, such as peptides, were unreliable in these oligomer applications (see, Car et al., Phys. Rev. Lett., 1985, 55, 2471-2474; Siepmann et al., Mol. Phys., 1992, 75, 59-70; Martin et al., J. Phys. Chem., 1999, 103, 4508-4517; and Brooks et al., J. Comp. Chem., 1983, 4, 187-217). Several chemical structural series of compounds have been prepared. See, for example, International Publication No. WO 2002/100295, which is incorporated herein by reference in its entirety. The compounds described herein can be prepared in a similar manner. Molecular dynamic and coarse grain modeling programs can be used for a design approach. See, for example, U.S. Application Publication No. 2004-0107056, and U.S. Application Publication No. 2004-0102941, each of which is incorporated herein by reference in its entirety.

After verifying the suitability of the force field by comparing computed predictions of the structure and thermodynamic properties to molecules that have similar torsional patterns and for which experimental data are available, the fitted torsions can then be combined with bond stretching, bending, one-four, van der Waals, and electrostatic potentials borrowed from the CHARMM (see, Brooks et al., J. Comp. Chem., 1983, 4, 187-217) and TraPPE (Martin et al., J. Phys. Chem., 1999, 103, 4508-4517; and Wick et al., J. Phys. Chem., 2000, 104, 3093-3104) molecular dynamics force fields. To identify conformations that can adopt periodic folding patterns with polar groups and apolar groups lined up on the opposite sides, initial structures can be obtained with the Gaussian package (see, Frisch et al., Gaussian 98 (revision A.7) Gaussian Inc., Pittsburgh, Pa. 1998). Then, the parallelized plane-wave Car-Parrinello CP-MD (see, Car et al., Phys. Rev. Lett., 1985, 55, 2471-2474) program, (see, Róthlisberger et al., J. Chem. Phys., 1996, 3692-3700) can be used to obtain energies at the minimum and constrained geometries. The conformations of the compounds without side-chains can be investigated in the gas phase. Both MD and MC methods can be used to sample the conformations. The former is useful for global motions of the compound. With biasing techniques (see, Siepmann et al., Mol. Phys., 1992, 75, 59-70; Martin et al., J. Phys. Chem., 1999, 103, 4508-4517; and Vlugt et al., Mol. Phys., 1998, 94, 727-733), the latter allows efficient sampling for compounds with multiple local minimum configurations that are separated by relatively large barriers.

The potential conformations are examined for positions to attach pendant groups that will impart amphiphilic character to the secondary structure. Compounds selected from the gas phase studies with suitable backbone conformations and with side-chains at the optimal positions to introduce amphiphilicity can be further evaluated in a model interfacial system. n-hexane/water can be chosen because it is simple and cheap for calculations while it mimics well the lipid/water bilayer environment. Compound secondary structures that require inter-compound interactions can be identified by repeating the above-mentioned calculations using a periodically repeated series of unit cells of various symmetries (so called variable cell molecular dynamics or Monte Carlo technique) with or without solvent. The results of these calculations can guide the selection of candidates for synthesis.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginally, by inhalation, by depot injections, or by implants. The mode of administration can depend on the pathogen or microbe to be targeted. The selection of the specific route of administration can be selected or adjusted by the clinician according to methods known to the clinician to obtain the desired clinical response.

In some embodiments, it may be desirable to administer one or more compounds, or a pharmaceutically acceptable salt thereof, locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as sialastic membranes, or fibers.

The compounds described herein can be administered either alone or in combination (concurrently or serially) with other pharmaceuticals. For example, the compounds can be administered in combination with another anti-heparin agent, including, but not limited to, protamine molecules. The compounds can also be administered in combination with other anti-cancer or anti-neoplastic agents, or in combination with other cancer therapies other than chemotherapy, such as, for example, surgery or radiotherapy. In some embodiments, the compounds described herein can also be administered in combination with (i.e., as a combined formulation or as separate formulations) with antibiotics, such as, for example: 1) protein synthesis inhibitors including, but not limited to, amikacin, anisomycin, apramycin, azithromycin, blasticidine S, brefeldin A, butirosin, chloramphenicol, chlortetracycline, clindamycin, clotrimazole, cycloheximide, demeclocycline, dibekacin, dihydrostreptomycin, doxycycline, duramycin, emetine, erythromycin, fusidic acid, G 418, gentamicin, helvolic acid, hygromycin B, josamycin, kanamycin, kirromycin, lincomycin, meclocycline, mepartricin, midecamycin, minocycline, neomycin, netilmicin, nitrofurantoin, nourseothricin, oleandomycin, oxytetracycline, paromomycin, puromycin, rapamycin, ribostamycin, rifampicin, rifamycin, rosamicin, sisomicin, spectinomycin, spiramycin, streptomycin, tetracycline, thiamphenicol, thiostrepton, tobramycin, tunicamycin, tylosin, viomycin, and virginiamycin; 2) DNA synthesis interfering agents including, but not limited to, camptothecin, 10-deacetylbaccatin III, azacytidine, 7-aminoactinomycin D, 8-quinolinol, 9-dihydro-13-acetylbaccatin III, aclarubicin, actinomycin D, actinomycin I, actinomycin V, bafilomycin A1, bleomycin, capreomycin, chromomycin, cinoxacin, ciprofloxacin, cis-diammineplatinum(II) dichloride, coumermycin A1, L(+)-lactic acid, cytochalasin B, cytochalasin D, dacarbazine, daunorubicin, distamycin A, doxorubicin, echinomycin, enrofloxacin, etoposide, flumequine, formycin, fumagillin, ganciclovir, gliotoxin, lomefloxacin, metronidazole, mithramycin A, mitomycin C, nalidixic acid, netropsin, nitrofurantoin, nogalamycin, nonactin, novobiocin, ofloxacin, oxolinic acid, paclitaxel, phenazine, phleomycin, pipemidic acid, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, trimethoprim, tubercidin, 5-azacytidine, cordycepin, and formycin A; 3) cell wall synthesis interfering agents including, but not limited to, (+)-6-aminopenicillanic acid, 7-Aminodesacetoxycephalosporanic acid, amoxicillin, ampicillin, azlocillin, bacitracin, carbenicillin, cefaclor, cefamandole, cefazolin, cefmetazole, cefoperazone, cefotaxime, cefsulodin, ceftriaxone, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillic, penicillin G, phenethicillin, phenoxymethylpenicillinic acid, phosphomycin, pipemidic acid, piperacillin, ristomycin, and vancomycin; 4) cell membrane permeability interfering agents (ionophores) including, but not limited to, 2-mercaptopyridine, 4-bromocalcimycin A23187, alamethicin, amphotericin B, calcimycin A23187, chlorhexidine, clotrimazole, colistin, econazole, hydrocortisone, filipin, gliotoxin, gramicidin A, gramicidin C, ionomycin, lasalocid A, lonomycin A, monensin, N-(6-aminohexyl)-5-chloro-1-naphthalenesulfonamide, narasin, nigericin, nisin, nonactin, nystatin, phenazine, pimaricin, polymyxin B, DL-penicillamine, polymyxin B, praziquantel, salinomycin, surfactin, and valinomycin; 5) enzyme inhibitors including, but not limited to, (+)-usnic acid, (±)-miconazole, (S)-(+)-camptothecin, 1-deoxymannojirimycin, 2-heptyl-4-hydroxyquinoline N-oxide, cordycepin, 1,10-phenanthroline, 6-diazo-5-oxo-L-norleucine, 8-quinolinol, antimycin, antipain, ascomycin, azaserine, bafilomycin, cerulenin, chloroquine, cinoxacin, ciprofloxacin, mevastatin, concanamycin A, concanamycin C, coumermycin A1, L(+)-lactic acid, cyclosporin A, econazole, enrofloxacin, etoposide, flumequine, formycin A, furazolidone, fusaric acid, geldanamycin, gliotoxin, gramicidin A, gramicidin C, herbimycin A, indomethacin, irgasan, lomefloxacin, mycophenolic acid, myxothiazol, N-(6-aminohexyl)-5-chloro-1-naphthalenesulfonamide, nalidixic acid, netropsin, niclosamide, nikkomycin, N-methyl-1-deoxynojirimycin, nogalamycin, nonactin, novobiocin, ofloxacin, oleandomycin, oligomycin, oxolinic acid, piericidin A, pipemidic acid, radicicol, rapamycin, rebeccamycin, sinefungin, staurosporine, stigmatellin, succinylsulfathiazole, succinylsulfathiazole, sulfadiazine, sulfadimethoxine, sulfaguanidine, sulfamethazine, sulfamonomethoxine, sulfanilamide, sulfaquinoxaline, sulfasalazine, sulfathiazole, triacsin C, trimethoprim, and vineomycin A1; and 6) membrane modifiers including, but not limited to, paracelsin.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above. The selection of the specific dose regimen can be selected or adjusted or titrated by the clinician according to methods known to the clinician to obtain the desired clinical response.

The amount of a compound described herein that will be effective in the antagonization of a heparin can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about 10 mg to about 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

In some embodiments, the salicylamide compound is present as a unit dose amount from about 5 mg to about 60 mg, and the histamine blocking agent is present as a unit dose amount from about 5 mg to about 50 mg. In some embodiments, the salicylamide compound is present as a unit dose amount from about 10 mg to about 55 mg, and the histamine blocking agent is present as a unit dose amount from about 10 mg to about 45 mg. In some embodiments, the salicylamide compound is present as a unit dose amount from about 15 mg to about 50 mg, and the histamine blocking agent is present as a unit dose amount from about 15 mg to about 40 mg. In some embodiments, the salicylamide compound is present as a unit dose amount from about 20 mg to about 45 mg, and the histamine blocking agent is present as a unit dose amount from about 20 mg to about 35 mg. In some embodiments, the salicylamide compound is present as a unit dose amount from about 25 mg to about 40 mg, and the histamine blocking agent is present as a unit dose amount from about 25 mg to about 30 mg. In some embodiments, the salicylamide compound is present as a unit dose amount from about 30 mg to about 35 mg, and the histamine blocking agent is present as a unit dose amount from about 25 mg to about 30 mg.

The compounds described herein can be formulated for parenteral administration by injection, such as by bolus injection or continuous infusion. The compounds can be administered by continuous infusion subcutaneously over a period of about 5 minutes to about 24 hours. The compounds can be administered by continuous infusion subcutaneously over a period of about 5 minutes to about 1 hour. The compounds can be administered by continuous infusion subcutaneously over a period of about 5 minutes to about 45 minutes. The compounds can be administered by continuous infusion subcutaneously over a period of about 5 minutes to about 30 minutes. The compounds can be administered by continuous infusion subcutaneously over a period of about 5 minutes to about 20 minutes. The compounds can be administered by continuous infusion subcutaneously over a period of about 5 minutes to about 15 minutes. The compounds can be administered by continuous infusion subcutaneously over a period of about 5 minutes to about 10 minutes. The compounds can be administered by continuous infusion subcutaneously over a period of about 10 minutes to about 15 minutes.

Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides. The compounds described herein can also be formulated in vaginal compositions such as vaginal creams, suppositories, pessaries, vaginal rings, and intrauterine devices.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In yet another embodiment, the compounds can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

It is also known in the art that the compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the excipient is a multi-component system chosen from 20% w/v propylene glycol in saline, 30% w/v propylene glycol in saline, 40% w/v propylene glycol in saline, 50% w/v propylene glycol in saline, 15% w/v propylene glycol in purified water, 30% w/v propylene glycol in purified water, 50% w/v propylene glycol in purified water, 30% w/v propylene glycol and 5 w/v ethanol in purified water, 15% w/v glycerin in purified water, 30% w/v glycerin in purified water, 50% w/v glycerin in purified water, 20% w/v Kleptose in purified water, 40% w/v Kleptose in purified water, and 25% w/v Captisol in purified water. In some embodiments, the excipient is chosen from 50% w/v propylene glycol in purified water, 15% w/v glycerin in purified water, 20% w/v Kleptose in purified water, 40% w/v Kleptose in purified water, and 25% w/v Captisol in purified water. In some embodiments, the excipient is chosen from 20% w/v Kleptose in purified water, 20% w/v propylene glycol in purified water, and 15% w/v glycerin in purified water.

In some embodiments, the composition comprises 50 mg/mL of compound in 20% w/v Kleptose in purified water.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In one embodiment, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

The compositions of the present disclosure can take the form of a liquid or solid, including, e.g., but not limited to, a solution, a suspension, an emulsion, a gel, an ointment, or a solid article that can be inserted in a suitable location in the eye or ear.

In some embodiments, a composition of the present disclosure is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can acceptable glycols, polyglycols, e.g., polyethylene glycol 400 (PEG-400), and glycol ethers.

Suitable solubilizing agents for solution and solution/suspension compositions are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Applications of cyclodextrins have been reviewed in Rajewski et al., Journal of Pharmaceutical Sciences, 1996, 85, 1155-1159. An acceptable cyclodextrin can optionally be present in a composition at a concentration from about 1 to about 200 mg/ml, from about 5 to about 100 mg/ml, or from about 10 to about 50 mg/ml.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers. However, in some embodiments, compositions do not contain substantial amounts of solid particulate matter, whether of the anti-microbial polymer or oligomer active agent, an excipient, or both, as solid particulate matter, if present, can cause discomfort and/or irritation of a treated eye.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts can be included in the compositions of the disclosure in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally an acceptable xanthine derivative such as caffeine, theobromine or theophylline can be included in the compositions, e.g., as disclosed in U.S. Pat. No. 4,559,343. Inclusion of the xanthine derivative can reduce ocular discomfort associated with administration of the composition.

Optionally one or more acceptable surfactants, preferably nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

One or more lubricating agents can also be included optionally in the compositions to promote lacrimation or as a “dry eye” medication. Such agents include, but are not limited to, polyvinyl alcohol, methylcellulose, hydroxypropyl methylcellulose, polyvinylpyrrolidone, and the like. It will be understood that promotion of lacrimation is beneficial in the present disclosure only where lacrimation is naturally deficient, to restore a normal degree of secretion of lacrimal fluid. Where excessive lacrimation occurs, residence time of the composition in the eye can be reduced.

Compositions of the present disclosure typically include a combination of one or more of the optional excipients listed above. For example, in some embodiments, the composition can optionally further comprise glycerin in an amount from about 0.5% to about 5%, from about 1% to about 2.5%, or from about 1.5% to about 2% by weight. Glycerin can be useful to increase viscosity of the composition and for adjustment of osmolality. Independently of the presence of glycerin, the composition can also further comprise a cyclodextrin, such as hydroxypropyl-β-cyclodextrin, in an amount from about 0.5% to about 25% by weight, as a solubilizing agent, and an antimicrobially effective amount of a preservative, e.g., imidazolidinyl urea in an amount from about 0.03% to about 0.5%; methylparaben in an amount from about 0.015% to about 0.25%; propylparaben in an amount from about 0.005% to about 0.01%; phenoxyethanol in an amount from about 0.25% to about 1%; disodium EDTA in an amount from about 0.05% to about 0.2%; thimerosal in an amount from 0.001% to about 0.15%; chlorobutanol in an amount from about 0.1% to about 0.5%; and/or sorbic acid in an amount from about 0.05% to about 0.2%; all by weight.

Thus, e.g., in some embodiments, the composition is a sterile aqueous solution comprising one or more of the disclosed polymers or oligomers, glycerin, sodium bicarbonate, and, optionally, a preservative, in purified water.

The present disclosure also provides pharmaceutical packs or kits comprising one or more containers filled with one or more compounds or compositions described herein. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one compound described herein. In some embodiments, the kit comprises a compound described herein in a single injectable dosage form, such as a single dose within an injectable device such as a syringe with a needle.

In some embodiments, the compositions are administered with an additional anti-microbial agent, such as, e.g., an anti-bacterial, anti-fungal, or anti-viral agent. For example, the additional anti-microbial agent can be a second compound disclosed herein, or the additional anti-microbial agent can be another anti-microbial agent such as, for example, an antibiotic selected from the group consisting of aminoglycosides, cephalosporins, diaminopyridines, fluoroquinolones, sulfonamides and tetracyclines. Examples of useful antibiotics which can serve as additional anti-microbials include, but are not limited to, amikacin, azithromycin, cefixime, cefoperazone, cefotaxime, ceftazidime, ceftizoxime, ceftriaxone, chloramphenicol, ciprofloxacin, clindamycin, colistin, domeclocycline, doxycycline, erythromycin, gentamicin, mafenide, methacycline, minocycline, neomycin, norfloxacin, ofloxacin, oxytetracycline, polymyxin B, pyrimethamine, silver sulfadiazine, sulfacetamide, sulfisoxazole, tetracycline, tobramycin, and trimethoprim.

The anti-inflammatory agents can be steroidal or non-steroidal. Examples of suitable steroidal anti-inflammatory agents include, but are not limited to, dexamethasone; dexamethasone derivatives such as those disclosed in U.S. Pat. No. 5,223,492; rimexolone; prednisolone; fluorometholone; and hydrocortisone.

Examples of suitable non-steroidal anti-inflammatory agents include, but are not limited to, prostaglandin H synthetase inhibitors (Cos I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, tenoxicam and carprofen; cyclooxygenase type II selective inhibitors, such as vioxx, celecoxib, etodolac; PAF antagonists, such as apafant, bepafant, minopafant, nupafant and modipafant; PDE IV inhibitors, such as ariflo, torbafylline, rolipram, filaminast, piclamilast, cipamfylline, and roflumilast; inhibitors of cytokine production, such as inhibitors of the NFkB transcription factor; or other anti-inflammatory agents know to those skilled in the art.

Examples of suitable topical or regional anesthetic agents include, but are not limited to, benzocaine.

Examples of suitable anti-allergic agents include, but are not limited to, pemirolast, olopatadine, and the corticosteroids (prednisolone, fluorometholone, loteprenol and dexamthasone).

The additional medicament can be administered in co-therapy (including co-formulation) with the one or more salicylamide compounds. For example, in some embodiments, an composition of the present disclosure comprising one of the anti-microbial oligomer disclosed herein is administered in co-therapy with an anti-inflammatory agent, e.g., a glucocorticoid.

In some embodiments, the response of the treatment is monitored and the treatment regimen is adjusted if necessary in light of such monitoring.

The compositions, such as aqueous suspension compositions, can be packaged in single-dose non-reclosable containers. Such containers can maintain the composition in a sterile condition and thereby eliminate need for preservatives such as mercury-containing preservatives, which can sometimes cause irritation and sensitization of the eye. Alternatively, multiple-dose reclosable containers can be used, in which case it is preferred to include a preservative in the composition.

In some embodiments, the composition is an aqueous solution, suspension or solution/suspension which is administered in the form of eye drops. In these embodiments, a desired dosage of the active agent can be administered by means of a suitable dispenser as a known number of drops into the eye. Examples of suitable dispensers are disclosed in International Patent Publication No. WO 96/06581.

In some embodiments, an effective concentration of the compound in the composition will generally be from about 0.01% to about 20% by weight (wt %) of the composition, from about 0.05% to about 10% by weight, from about 0.1% to about 8.0% by weight, from about 0.5% to about 5.0% by weight, from about 1.0% to about 5.0% by weight, or from about 2.0% to about 4.0% of the composition. For example, in compositions in the form of solid suspensions, such as ointments, an effective concentration of the antimicrobial polymer or oligomer will generally be from about 1% to about 5% by weight (wt %) of the composition.

The present disclosure also provides methods of antagonizing unfractionated heparin, low molecular weight heparin, or a heparin/low molecular weight heparin derivative in a mammal comprising administering any of the foregoing pharmaceutical compositions to the mammal

The present disclosure also provides methods of antagonizing unfractionated heparin, low molecular weight heparin, or a heparin/low molecular weight heparin derivative in a mammal comprising: administering one or more histamine blocking agents to the mammal; and administering a salicylamide compound to the mammal

In some embodiments, the salicylamide compound is a compound of Formula I:

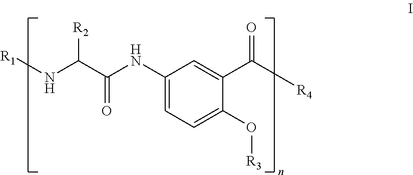

wherein: n is 2 to 10; R₁ is H or

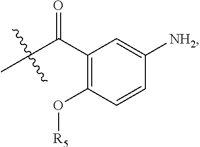

where R₅ is H or C₁ to C₉ straight or branched alkyl optionally substituted with one or more —NH₂, —N(CH₃)₂, or

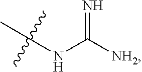 [SMILES: *NC(=N)N]

or a suitable substituent; each R₂ is, independently, C₁ to C₉ straight or branched alkyl optionally substituted with one or more —NH₂, —N(CH₃)₂, or

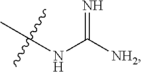 [SMILES: *NC(=N)N]

or a suitable substituent; each R₃ is, independently, C₁ to C₉ straight or branched alkyl optionally substituted with one or more —NH₂, —N(CH₃)₂, or

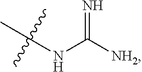 [SMILES: *NC(=N)N]

or a suitable substituent; and R₄ is OH, NH₂, or

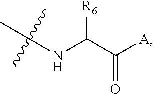

where A is OH or NH₂, and R₆ is H or C₁ to C₉ straight or branched alkyl optionally substituted with one or more —NH₂, —N(CH₃)₂, or

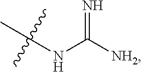 [SMILES: *NC(=N)N]

or a suitable substituent; or a pharmaceutically acceptable salt thereof.

In some embodiments, n is 3 to 8. In some embodiments, n is 3 to 5. In some embodiments, n is 3 or 4.

In some embodiments, R₁ is H.

In some embodiments, each R₂ is, independently, C₃ to C₅ straight or branched alkyl optionally substituted with one or more —NH₂ or

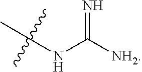 [SMILES: *NC(=N)N]

In some embodiments, each R₂ is, independently, C₃ or C₄ straight alkyl optionally substituted with one —NH₂ or

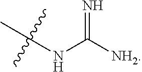 [SMILES: *NC(=N)N]

In some embodiments, each R₂ is, independently, C₃ or C₄ straight alkyl substituted with one —NH₂ or

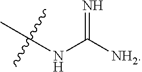 [SMILES: *NC(=N)N]

In some embodiments, each R₃ is, independently, C₁ to C₉ straight or branched alkyl. In some embodiments, each R₃ is, independently, C₁ to C₃ straight alkyl.

In some embodiments, R₄ is OH, NH₂, or

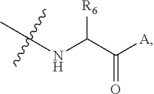

where A is NH₂, and R₆ is C₁ to C₉ straight or branched alkyl optionally substituted with one —NH₂, —N(CH₃)₂, or

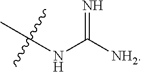 [SMILES: *NC(=N)N]

In some embodiments, R₄ is OH or NH₂.

In some embodiments, n is 3 to 5; R₁ is H; each R₂ is, independently, C₃ to C₅ straight alkyl optionally substituted with one —NH₂, —N(CH₃)₂, or

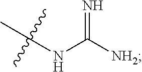 [SMILES: *NC(=N)N]

each R₃ is, independently, C₁ to C₃ straight alkyl optionally substituted with one —NH₂; and R₄ is OH or NH₂.

In some embodiments, n is 3 or 4; R₁ is H; each R₂ is, independently, C₃ or C₄ straight alkyl substituted with one —NH₂ or

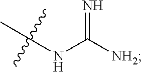 [SMILES: *NC(=N)N]

each R₃ is, independently, C₁ or C₂ alkyl; and R₄ is NH₂.

In some embodiments, the salicylamide compound is chosen from:

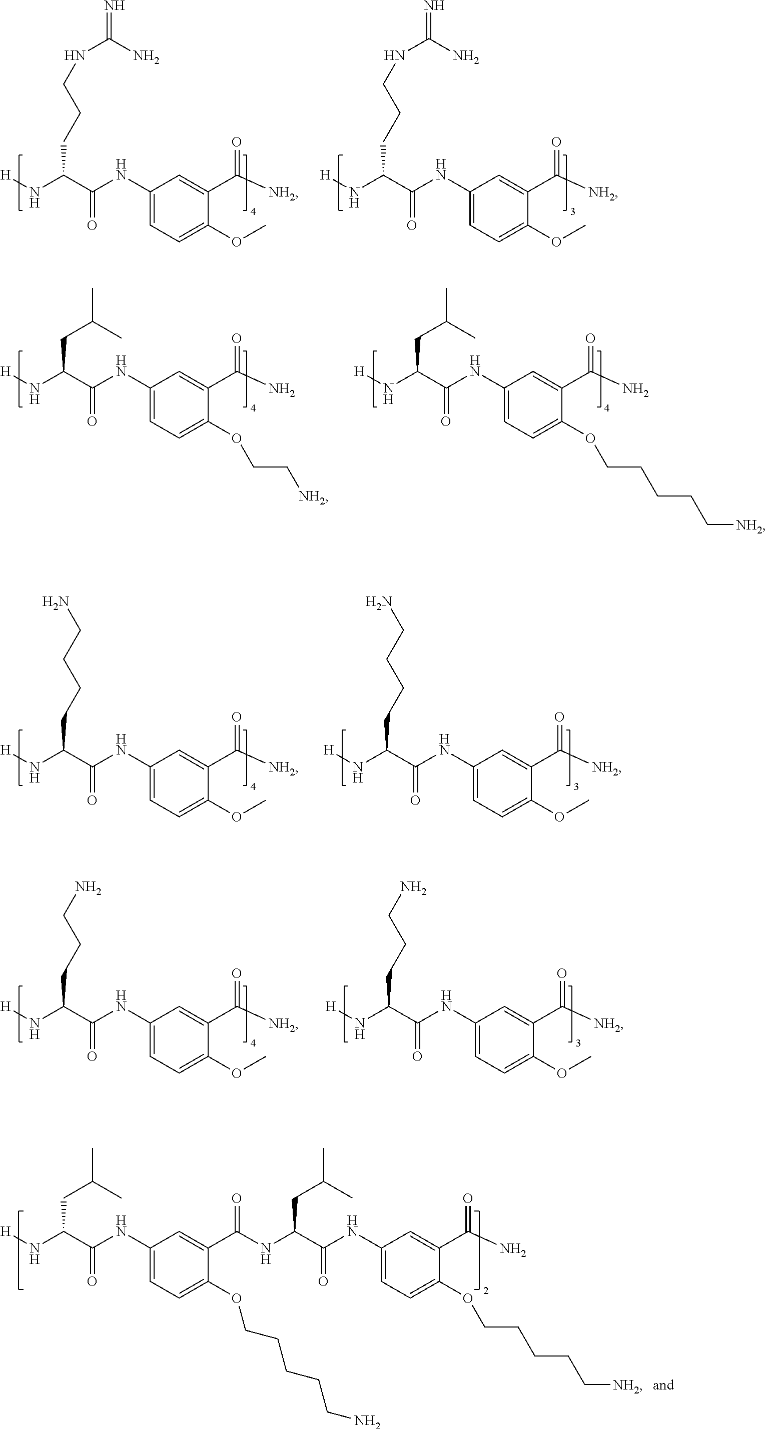

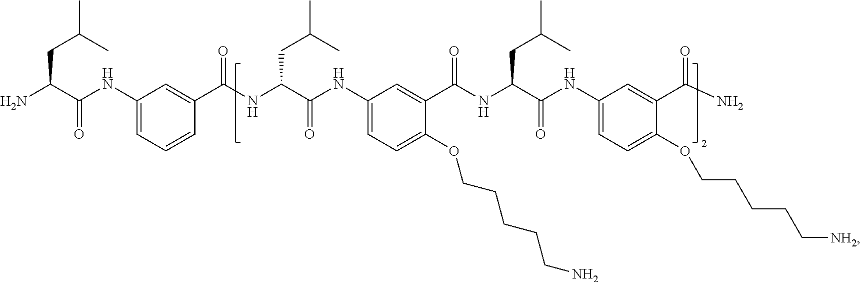

or a pharmaceutically acceptable salt thereof. In some embodiments, the salicylamide compound is

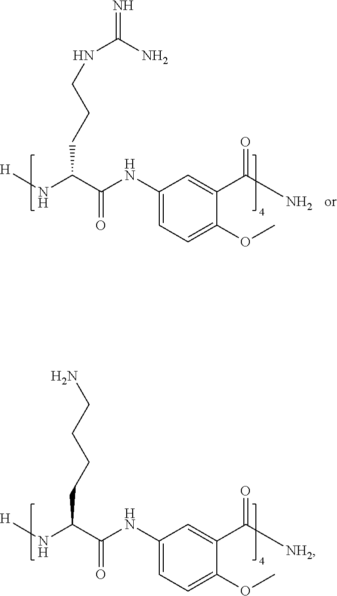

or a pharmaceutically acceptable salt thereof.

In some embodiments, the histamine blocking agent can be an H1-receptor and/or H2-receptor antagonist or is chosen from diphenhydramine (Benadryl), loratadine (Claritin), fexofenadine (Allegra), chlorpheniramine (Chlor-Tripalon), cimetidine (Tagamet), brompheniramine (Dimetane), dimenhydrinate (Gravol), promethazine (Phenergan), hydroxyzine (Atarax), cyproheptadine (Periactin), azatadine (Zadine), and cetirizine (Reactine), or a pharmaceutically acceptable salt thereof. In some embodiments, the histamine blocking agent is diphenhydramine. In some embodiments, a combination of two or more histamine blocking agents is used. In some embodiments, the combination is diphenhydramine and cimetidine.

In some embodiments, the salicylamide compound is

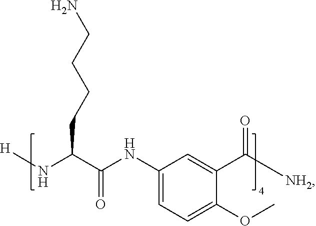

or a pharmaceutically acceptable salt thereof, and the histamine blocking agent is diphenhydramine, cimetidine, or a combination of diphenhydramine and cimetidine.

The compounds may be useful as anti-heparin agents (i.e., antagonizing the anticoagulant effect of an anticoagulant such as unfractionated heparin, low molecular heparin, and a derivative of heparin or low molecular heparin) in a number of applications. For example, compounds may be used therapeutically to antagonize the anticoagulant effect of an anticoagulant agent (for example unfractionated heparin, low molecular heparin, or a derivative of heparin or low molecular heparin), present in a mammal. The anticoagulant effect of the anticoagulant agent (for example unfractionated heparin, low molecular heparin, or a derivative of heparin or low molecular heparin) present in a mammal may be antagonized by administering to the mammal an effective amount of a compound or a pharmaceutically acceptable salt thereof, or a pharmaceutical composition comprising the same.

Natural heparins have polysaccharide chains of varying lengths, or molecular weights (including salts). Natural heparin has polysaccharide chains of molecular weight from about 5000 to over 40,000 Daltons. Low-molecular-weight heparins (LMWHs), in contrast, are fragments of unfractionated heparins, and have short chains of polysaccharide (including salts). LMWHs have an average molecular weight of less than 8000 Da and at least 60% of all chains have a molecular weight less than 8000 Da.

In some embodiments, the methods of the present disclosure can effectively antagonize the anticoagulant effect of unfractionated heparin. In some embodiments, the methods of the present disclosure can effectively antagonize the anticoagulant effect of a low molecular weight heparin such as enoxaparin. In some embodiments, the methods of the present disclosure can effectively antagonize the anticoagulant effect of a synthetically modified heparin derivative such as fondaparinux.

In some embodiments, the method of the present disclosure can antagonize greater than about 50%, greater than about 60%, greater than about 70%, greater than about 80%, greater than about 85%, greater than about 88%, greater than about 90%, greater than about 92%, greater than about 95%, greater than about 98%, greater than about 99%, greater than about 99.2%, greater than about 99.5%, greater than about 99.8%, or greater than about 99.9% of the anticoagulant effect of heparin (including, for example, unfractionated heparin, low molecular weight heparin, and synthetically modified heparin or low molecular heparin derivatives). In some embodiments, the compound or salt thereof used in the present disclosure antagonizes the anticoagulant effect of an anticoagulant agent (including, for example, unfractionated heparin, low molecular weight heparin, and synthetically modified heparin or low molecular heparin derivatives) more effectively than protamine.

In some embodiments, the compound or salt thereof used in the present disclosure binds to heparin (including, for example, unfractionated heparin, low molecular weight heparin, and synthetically modified heparin or low molecular heparin derivatives) with an EC₅₀ of less than about 100, less than about 90, less than about 80, less than about 70, less than about 60, less than about 50, less than about 40, less than about 30, less than about 20, less than about 15, less than about 10, less than about 5, less than about 2, less than about 1, less than about 0.9, less than about 0.8, less than about 0.7, less than about 0.6, less than about 0.5, less than about 0.4, less than about 0.3, less than about 0.2, less than about 0.1, less than about 0.09, less than about 0.08, less than about 0.07, less than about 0.06, less than about 0.05, less than about 0.02, less than about 0.01, less than about 0.001, less than about 0.0001, or less than about 0.00001 μg/mL.

In some embodiments, the compound or salt thereof used in the present disclosure binds to heparin (including, for example, unfractionated heparin, low molecular weight heparin, and synthetically modified heparin or low molecular heparin derivatives) with an EC₅₀ less than about 100, less than about 90, less than about 80, less than about 70, less than about 60, less than about 50, less than about 40, less than about 30, less than about 20, less than about 15, less than about 10, less than about 5, less than about 2, less than about 1, less than about 0.9, less than about 0.8, less than about 0.7, less than about 0.6, less than about 0.5, less than about 0.4, less than about 0.3, less than about 0.2, less than about 0.1, less than about 0.09, less than about 0.08, less than about 0.07, less than about 0.06, less than about 0.05, less than about 0.02, less than about 0.01, less than about 0.001, less than about 0.0001, or less than about 0.00001 μM.

In some embodiments, the compound or salt thereof used in the present disclosure binds to heparin (including, for example, unfractionated heparin, low molecular weight heparin, and synthetically modified heparin or low molecular heparin derivatives) with an EC₅₀ of less than that of protamine (including protamine salt such as protamine sulfate).

In some embodiments, the compound or salt thereof used in the present disclosure can effectively antagonize the anticoagulant effect of an anticoagulant agent (including, for example, unfractionated heparin, low molecular weight heparin, and synthetically modified heparin or low molecular heparin derivatives) with a dosage of less than about 10, less than about 9, less than about 8, less than about 7, less than about 6, less than about 5, less than about 4, less than about 3, less than about 2, or 1 equivalent (by weight) to the heparin.

In some embodiments, the compound or salt thereof used in the present disclosure can effectively antagonize the anticoagulant effect of an anticoagulant agent (including, for example, unfractionated heparin, low molecular weight heparin, and synthetically modified heparin or low molecular heparin derivatives) through antagonizing the AT activity of the heparin, the anti-factor Xa activity of the heparin, the anti-factor IIa activity of the heparin, or any combination thereof.

In some embodiments, the method of the present disclosure can rapidly antagonize the anticoagulant effect of an anticoagulant agent (including, for example, unfractionated heparin, low molecular weight heparin, and synthetically modified heparin or low molecular heparin derivatives), for example, antagonize (or neutralize) greater than about 40%, greater than about 50%, greater than about 60%, greater than about 70%, greater than about 80, greater than about 90%, greater than about 95%, greater than about 98%, greater than about 99%, or greater than about 99.5% of the anticoagulant effect of the heparin in less than about 30, less than about 20, less than about 15, less than about 10, less than about 8, less than about 5, less than about 2, less than about 1, less than about 0.9, less than about 0.8, less than about 0.7, less than about 0.6, less than about 0.5, less than about 0.4, less than about 0.3, less than about 0.2, or less than about 0.1 minute.

In some embodiments, after the anticoagulant effect of heparin in a mammal during anticoagulant therapy is antagonized (for example, by 80% or more) by methods of the present disclosure, a new dose of heparin can effectively restore the anticoagulant therapy, for example, greater than about 80% or 90% of the anticoagulant effect of heparin of the new dose can be achieved in less than about 20, less than about 15, less than about 10, less than about 8, less than about 5, less than about 2, or less than about 1 minute.

In some embodiments, the present disclosure provides methods for antagonizing the anticoagulant effect of heparin with low or no toxicity, hemodynamic and/or hematological adverse side effects. In some embodiments, the methods have low or no side effects associated with use of protamine such as one or more selected from systemic vasodilation and hypotension, bradycardia, pulmonary artery hypertension, pulmonary vasoconstriction, thrombocytopenia, and neutropenia. In some embodiments, the methods have low or no side effects associated with use of protamine such as anaphylactic-type reactions involving both nonimmunogenic and immunogenic-mediated pathways. In some embodiments, the compounds and/or the salts have low or no antigenicity and/or immunogenicity comparing to those of protamine molecules. In some embodiments, the present methods for antagonizing the anticoagulant effect of heparin can preserve hemodynamic stability, such as during and/or following infusion.

In some embodiments, the present methods for antagonizing the anticoagulant effect of heparin can be used in a patient who receives anticoagulant therapy, for example, who uses fondaparinux for the prophylaxis of deep vein thrombosis following hip repair/replacement, knee replacement and abdominal surgery; uses UFH or LMWH for coronary bypass surgery; or uses UFH or LMWH during and/or following blood infusion.

In some embodiments, the unfractionated heparin is antagonized. In some embodiments, the low molecular weight heparin is antagonized. In some embodiments, the low molecular weight heparin is enoxaparin, reviparin, or tinzaparin. In some embodiments, the heparin/low molecular weight heparin derivative is antagonized. In some embodiments, the heparin/low molecular weight heparin derivative is fondaparinux. In some embodiments, the mammal is a human.

In some embodiments, the weight ratio of the compound, or pharmaceutically acceptable salt thereof, to be administered, to the unfractionated heparin, low molecular weight heparin, or heparin/low molecular weight heparin derivative is less than about 10:1. In some embodiments, the weight ratio of the compound, or pharmaceutically acceptable salt thereof, to be administered, to the unfractionated heparin, low molecular weight heparin, or heparin/low molecular weight heparin derivative is less than about 5:1, less than about 10:1, less than about 25:1, or less than about 30:1. In some embodiments, the weight ratio of the compound, or pharmaceutically acceptable salt thereof, to be administered, to the unfractionated heparin, low molecular weight heparin, or heparin/low molecular weight heparin derivative is from about 1:1 to about 5:1, from about 1:1 to about 10:1, or from about 1:1 to about 25:1.

In some embodiments, the salicylamide compound is administered from about 10 minutes to about 40 minutes after administration of the histamine blocking agent. In some embodiments, the salicylamide compound is administered from about 15 minutes to about 30 minutes after administration of the histamine blocking agent. In some embodiments, the salicylamide compound is administered from about 15 minutes to about 20 minutes after administration of the histamine blocking agent.

In some embodiments, the histamine blocking agent is administered to the mammal as an intravenous infusion.

In some embodiments, the salicylamide compound is administered to the mammal as an intravenous infusion.

In some embodiments, from about 5 mg to about 50 mg of the histamine blocking agent is administered to the mammal. In some embodiments, from about 10 mg to about 45 mg of the histamine blocking agent is administered to the mammal. In some embodiments, from about 15 mg to about 40 mg of the histamine blocking agent is administered to the mammal. In some embodiments, from about 20 mg to about 35 mg of the histamine blocking agent is administered to the mammal. In some embodiments, from about 25 mg to about 30 mg of the histamine blocking agent is administered to the mammal. In some embodiments, about 25 mg of the histamine blocking agent is administered to the mammal.

In some embodiments, from about 5 mg to about 60 mg of the salicylamide compound is administered to the mammal. In some embodiments, from about 10 mg to about 55 mg of the salicylamide compound is administered to the mammal. In some embodiments, from about 15 mg to about 50 mg of the salicylamide compound is administered to the mammal. In some embodiments, from about 20 mg to about 45 mg of the salicylamide compound is administered to the mammal. In some embodiments, from about 25 mg to about 40 mg of the salicylamide compound is administered to the mammal. In some embodiments, from about 30 mg to about 35 mg of the salicylamide compound is administered to the mammal.

In some embodiments, the low molecular weight heparin is enoxaparin, reviparin, or tinzaparin.

In some embodiments, the heparin/low molecular weight heparin derivative is fondaparinux.

The present disclosure also provides compositions (such as those described herein) for antagonizing unfractionated heparin, low molecular weight heparin, or a heparin/low molecular weight heparin derivative.

The present disclosure also provides compositions (such as those described herein) for use in the manufacture of a medicament for antagonizing unfractionated heparin, low molecular weight heparin, or a heparin/low molecular weight heparin derivative.

The present disclosure also provides use of compositions (such as those described herein) for antagonizing unfractionated heparin, low molecular weight heparin, or a heparin/low molecular weight heparin derivative.

The present disclosure also provides use of compositions (such as those described herein) for the manufacture of a medicament for antagonizing unfractionated heparin, low molecular weight heparin, or a heparin/low molecular weight heparin derivative.

In order that the disclosure disclosed herein may be more efficiently understood, examples are provided below. It should be understood that these examples are for illustrative purposes only and are not to be construed as limiting the disclosure in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Clotting and Amidolytic Assays

aPTT clotting Assay: Unfractionated heparin is mixed with plasma at a final concentration of 0.4 U/mL (or concentration which increases aPTT time to between 120 and 300 seconds). Different concentrations of test compound are added (typically 0.15 to 20 μg/mL range). The ACL Elite Hemostasis analyzer (Beckman Coulter™) is used to add aPTT reagent (HemoslL SynthASil) to supplemented plasma. Clotting is initiated by addition of CaCl₂ and time to clot is recorded. EC₅₀ values are determined using a curve fit program (GraphPad Prism 5).

FXa Amidolytic Assay: LMWH (enoxaparin or tinzaparin) at final concentrations of 0.1 μg/mL, UFH at final concentrations of 0.03 units/mL, or fondaparinux at a final concentration of 0.02 μg/mL (or concentration which fully inhibits factor Xa) is combined with human antithrombin at a final concentration of 0.036 units/mL. Two μL of test agent are added (range between 0.01 and 23 μg/mL) and incubated for 5 minutes at 23° C. Bovine Factor Xa was added to a final concentration of 0.636 nkat/mL and incubated for a further 10 minutes at 23° C. Using a SpectraMax 250 (Molecular Devices, Inc.) and SoftMax Pro V.5 software, the plate is read every 30 seconds for 4 minutes, with a 10 second shaking before first read and maximum interval shaking. Fit curve to report an EC₅₀ (50% reversal of anticoagulant effects) value for each compound:

P(Cₚ)=1/[1+(K/Cₚ)ⁿ].

### Example 2

### In Vivo Neutralization of Unfractionated Heparin in the Rat

Male Sprague-Dawley are obtained from Charles River Laboratories, Raleigh. They are nine-weeks-old at the start of the study and their weights range from 279-334 g. Rats are pre-treated with UFH administered by IV injection in a tail vein at 100 U/kg in a dose volume of 1 mL/kg. The rats are then treated with a single IV injection of saline, protamine or the appropriate test compound at doses of 0.25, 0.5 and 1.0 mg/kg. All treatments are dosed in a volume of 1 mL/kg. Blood is collected via the orbital sinus from three rats per group at the following time points after treatment: predose, 1, 3, 10, 30 and 60 minutes. At each time point, 1 mL of blood is collected from each animal into a single tube. The blood is analyzed using an AMEX Destiny Plus Coagulation Analyzer for activated partial thromboplastintime (APTT) and anti-Factor Xa.

### Example 3

### In Vivo Neutralization of Enoxaparin in the Rat

Compounds are tested for their ability to neutralize enoxaparin coagulation inhibition in rats. Male Sprague-Dawley rats are used in this study (Charles River Laboratories). They are ten-weeks-old at the start of the study and their weights range from 319-362 g. Enoxaparin (2 mg/kg) is administered by IV injection to groups of six rats. After 3 minutes, saline, protamine or a test compound is administered by IV injection. Blood is collected before dosing with enoxaparin, and at 1, 3, 10, 30 and 60 minutes after dosing with the standard and test compounds. All treatments are dosed in a volume of 1 mL/kg. Blood is collected via the orbital sinus from three rats per group. At each time point, 1 mL of blood is collected from each animal into a single tube. The blood is analyzed using an AMEX Destiny Plus Coagulation Analyzer for activated partial thromboplastin time (APTT) and anti-Factor Xa (low-molecular weight).

### Example 4

### Normalization of Enoxaparin-Extended Bleeding Times in a Rat Tail Transection Model

Studies are performed to examine effects on extended bleeding times caused by enoxaparin treatment. Male Sprague Dawley rats (Charles River) are administered 2 mg/kg enoxaparin by IV injection in the tail vein, followed 3 minutes later by test agent (IV, tail vein) at 2 and 5 mg/kg doses. Tails are then rapidly transected and bleeding time onto an absorbent pad was determined.

### Example 5

### In Vivo Neutralization of Fondaparinux in the Rat

Compounds are selected to test fondaparinux neutralization in vivo. Rats are pre-treated with fondaparinux administered by IV injection at 0.5 mg/kg. The rats are treated with a single IV injection of saline, protamine or the compound. Blood is collected via the orbital sinus from three rats per group at the following time points: pre-dose, 1, 3, 10, 30 and 60 minutes. Plasma samples are prepared for analysis of anti-factor Xa activity using an AMEX Destiny Plus Coagulation Analyzer.

### Example 6

### FXa Chromogenic Assay (Absence of Plasma)

Human antithrombin is mixed with an anticoagulant agent (a LMWH or fondaparinux); final concentrations are 0.22 μg/mL for the LMWHs and 0.07 μg/mL for fondaparinux. Different concentrations of a test compound are added (typically 0.07 to 9 μg/mL range) followed by factor Xa and substrate (S-2765). Absorbance is read every 30 seconds over a 4 minute period in a SpectraMax 250 instrument (Molecular Devices, Inc.). EC₅₀ values are determined by a curve-fit program (SoftMax Pro) using the following formula:

P(Cₚ)=1/[1+(K/Cₚ)ⁿ]

### Example 7

### FIIa (Thrombin) Chromogenic Assay (Absence of Plasma)

The procedure for measuring anti-FIIa activity is similar to that for the anti-FXa assay except FIIa and S-2238 are used in place of FXa and S-2765, respectively.

### Example 8

### Clotting and Amidolytic Assays in Presence of Human Plasma

Eight parts of pooled human plasma is supplemented with 1 part LMWH or UFH at final concentrations of 4 μg/mL, or fondarinux at a final concentration of 1.25 μg/mL. One μL sample of test agent is then added to 9 μL of supplemented plasma (test agent concentration ranges=0.156 to 20 μg/mL) and mixed. The supplemented plasmas are analyzed immediately in clotting and amidolytic assays as described below. All samples are performed in duplicate.

aPTT Clotting Assay. Supplemented plasma is added to aPTT reagent (activated partial thromboplastin time reagent) (activator) in fibrometer. Clotting is initiated by addition of CaCl₂ and time to clot was recorded.

HepTest Clotting Assay. Factor Xa is added to supplemented plasma in a fibrometer and incubated for 120 seconds. Recalmix is added and time to clot was recorded.

Thrombin time (TT) Clotting Assay. Human thrombin is added to supplemented plasma in a fibrometer and time to clot was recorded.

FXa Amidolytic Assay: Bovine factor Xa is added to supplemented plasma and incubated for 5 minutes at 37° C. Spectrozyme FXa substrate is added and the optical density change at 405 nm is measured for 30 seconds. % factor Xa inhibition is calculated using the following equation:

%Inhibition=[(OD_{baseline}−ODₛₐₘₚₗₑ)/OD_{baseline}]100.

FXa Amidolytic Assay: LMWH (enoxaparin or tinzaparin) at final concentrations of 0.1 μg/mL, UFH at final concentrations of 0.03 units/mL, or fondaparinux at a final concentration of 0.02 μg/mL (or concentration which fully inhibits factor Xa) is combined with human antithrombin at a final concentration of 0.036 units/mL. Two μL of test agent are added (range between 0.01 and 23 μg/mL) and incubated for 5 minutes at 23° C. Bovine Factor Xa was added to a final concentration of 0.636 nkat/mL and incubated for a further 10 minutes at 23° C. Using a SpectraMax 250 (Molecular Devices, Inc.) and SoftMax Pro V.5 software, the plate is read every 30 seconds for 4 minutes, with a 10 second shaking before first read and maximum interval shaking. Fit curve to report an EC₅₀ (50% reversal of anticoagulant effects) value for each compound:

P(Cₚ)=1/[1+(K/Cₚ)ⁿ].

FIIa Amidolytic Assay. Human thrombin is added to supplemented plasma and incubated for 1 minute at 37° C. Spectrozyme TH substrate is added and the optical density change at 405 nm is measured for 30 seconds in a SpectraMax 250 instrument. % factor IIa inhibition is calculated using the following equation:

%Inhibition=[(OD_{baseline}−ODₛₐₘₚₗₑ)/OD_{baseline}]×100.

### Example 9

### Heparin-Binding Activity

The heparin (unfractionated) preparations are tyramine end-labeled and radiolabeled with ¹²⁵Iodine to a specific activity of 1−2.5×10⁷ cpm/μg. Increasing concentrations of a test agent (protamine or an exemplary compound provided herein) are added to individual wells across a 1% agarose gel in 125 mM sodium acetate, 50 mM MOPSO (3-(n-morpholino)-2-hydroxypropanesulfonic acid), pH 7.0). The radio-labeled heparin is added to a closely neighboring upper well and electrophoresed through the test agent wells. Heparin binding is visualized on the dried gel using a Phosphorimager. The dissociation constant (Kd) is calculated from the test agent concentration (n=3) at which the polysaccharide is half-shifted between its fully mobile position at low concentrations of test agent and its fully retarded position at saturating concentrations of test agent according to the methods of Lee and Lander (See Lee, M. K. and Lander, A. D., “Analysis of affinity and structural selectivity in the binding of proteins to glycosaminoglycans: development of a sensitive electrophoretic approach” Proc. Natl. Acad. Sci. USA, 1991, 88, 2768-2772).

### Example 10

### In Vivo Neutralization of Unfractionated Heparin in the Rat

The male Sprague-Dawley rats used in this study are obtained from Charles River Laboratories, Raleigh. They are nine-weeks-old at the start of the study and their weights range from 279-334 g. Rats are pre-treated with UFH administered by IV injection in a tail vein at 100 U/kg in a dose volume of 1 mL/kg. The rats are then treated with a single IV injection of saline, protamine or the appropriate test compound at doses of 0.25, 0.5 and 1.0 mg/kg. All treatments are dosed in a volume of 1 mL/kg. Blood is collected via the orbital sinus from three rats per group at the following time points after treatment: predose, 1, 3, 10, 30 and 60 minutes. At each time point, 1 mL of blood is collected from each animal into a single tube. The blood is analyzed using an AMEX Destiny Plus Coagulation Analyzer for activated partial thromboplastintime (APTT) and anti-Factor Xa.

### Example 11

### In Vivo Neutralization of Enoxaparin in the Rat

Compounds are tested for their ability to neutralize enoxaparin coagulation inhibition in rats. Male Sprague-Dawley rats are used in this study (Charles River Laboratories). They are ten-weeks-old at the start of the study and their weights range from 319-362 g. Enoxaparin (2 mg/kg) is administered by IV injection to groups of six rats. After 3 minutes, saline, protamine or a test compound is administered by IV injection. Blood is collected before dosing with enoxaparin, and at 1, 3, 10, 30 and 60 minutes after dosing with the standard and test compounds. All treatments are dosed in a volume of 1 mL/kg. Blood is collected via the orbital sinus from three rats per group. At each time point, 1 mL of blood is collected from each animal into a single tube. The blood is analyzed using an AMEX Destiny Plus Coagulation Analyzer for activated partial thromboplastin time (APTT) and anti-Factor Xa (low-molecular weight).

### Example 12

### Normalization of Enoxaparin-Extended Bleeding Times in a Rat Tail Transection Model

Male Sprague Dawley rats (Charles River) are administered 2 mg/kg enoxaparin by IV injection in the tail vein, followed 3 minutes later by test agent (IV, tail vein) at 2 and 5 mg/kg doses. Tails are then rapidly transected and bleeding time onto an absorbant pad is determined

### Example 13

### In Vivo Neutralization of Fondaparinux in the Rat

Compounds are selected to test fondaparinux neutralization in vivo. Rats are pre-treated with fondaparinux administered by IV injection at 0.5 mg/kg. The rats are then treated with a single IV injection of saline, protamine or the compound. Blood is collected via the orbital sinus from three rats per group at the following time points: pre-dose, 1, 3, 10, 30 and 60 minutes. Plasma samples are prepared for analysis of anti-factor Xa activity using an AMEX Destiny Plus Coagulation Analyzer.

### Example 14

### Anti-Factor Xa Inhibition

The following example illustrates the effects of compounds of the present disclosure on anti-Factor Xa inhibition. To determine the anti-heparin activity of the compounds, an assay measuring the percent inhibition using a fixed concentration of compound or concentrations of compounds causing lysis of 50% of human red blood cells is used.

10 IU of anti-thrombin is dissolved in 10 mL of buffer, resulting in a 1 IU/mL stock solution (250×) of the anti-thrombin. The 1 IU/mL (250×) stock solution of anti-thrombin and a 336 mM stock solution of NaCl are diluted into a total volume of 50 μL buffer so that the final anti-thrombin concentration is 0.004 IU/sample well and the NaCl is 150 mM/sample well. 1 μL of the compound to be tested, final concentration 10 μg/mL (corresponding to 0.5 logarithmic antagonist dilution) is added to the sample well. The samples are mixed and allowed to incubate at room temperature for 20 minutes. 50 μL of factor Xa dissolved in buffer is added to the sample well to a final concentration of 0.14 knat/well (2 μL of the 7.1 knat/ml stock solution to a final sample well buffer volume of 100 μL). The samples are mixed and further incubated at room temperature for 10 minutes. 10 μL of a 4 mM stock solution of the substrate S-2765 is added to each sample well for a final concentration of 0.4 mM in each sample well. The samples are mixed and hydrolyses of the chromogenic substrate Z-D-Arg-Gly-Arg-pNA (S-2765), thus liberating the chromophoric group pNA (p-nitroaniline), is monitored at 405 nm. The samples are mixed every 30 seconds to maintain a uniform mixture. ThermoLabsystems Multiskan Spectrum spectrophotometer is used to measure the absorbance spectrums. The increase in absorbance is proportional to the enzyme (factor Xa) activity. The % inhibition of factor Xa is determined using a standard curve.

Anti-Factor Xa Inhibition: EC50. To determine the concentration of polycationic compound that causes about 50% lysis of human red blood cells, fixed heparin concentrations are used and different amounts of heparin antagonists are added.

### Example 15

### Histamine Release

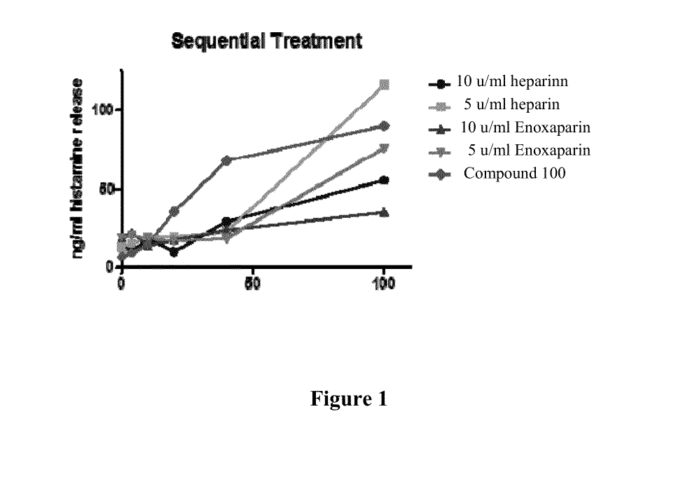

The subsequent assays demonstrate that when RBL cells are pre-treated with heparin or enoxaparin and Compound 100 is then added to the cells, the histamine release is abrogated to a degree consistent with the amount of free Compound 100, and is not unique to the chemical nature of the anticoagulant. These data are shown in the graph below. Note: RBL cells were exposed to heparin or enoxaparin for 5 minutes at 37° C. Compound 100 was added and incubated for an additional 5 minutes. Cell supernatant was removed and assayed for released histamine Results are shown in FIG. 1.

### Example 16

### Cardiovascular Effects of Compound 100 in Anesthetized Rats Pretreated with Heparin and Antagonists of Histamine or an Inhibitor of Nitric Oxide Production

A purpose of this study was to evaluate the role of histamine or nitric oxide (NO) on the hemodynamic effects of Compound 100 administered by a 10 minute IV infusion in rats by pretreatment with an H1 receptor blocker (diphenhydramine; DPH), an H2 receptor blocker (cimetidine; CIM), or an NO synthase inhibitor (L-NAME).

Surgically prepared animals (jugular vein catheter for test article administration and carotid artery catheter for blood pressure/heart rate measurement) were purchased from Charles River Laboratories, Raleigh, N.C. Animals were anesthetized on the day of experimentation with isoflurane (1.8-4%). Blood pressure and heart rate data were collected on a Grass Polygraph recorder. Groups of three or four animals were administered two pretreatments followed by the administration of the vehicle or Compound 100. Dose groups are described in Table 1. Treatment 1 was administered at T=−15 minutes as an intravenous administration of saline, cimetidine or L-Name, a subcutaneous dose of diphenhydramine or a combination of diphenhydramine (s.c.) and cimetidine (i.v.). Saline or heparin were administered at T=−3 minutes by intravenous administration (Treatment 2). Treatment 3 was Compound 100 or sterile water (vehicle) which was administered by a 10 minute intravenous infusion. Blood pressure and heart rate were recorded prior to Treatment 1, 5 and 10 minutes after Treatment 1, 2 minutes following Treatment 2, and at 1, 15, 25, 40, and 70 minutes following Treatment 3. Each recording interval was approximately 1 minute long.

TABLE 1
Group Assignments and Dose Levels

| | | | | | Treatment 3 |
|---|---|---|---|---|---|
| Group | (n) | Treatment 1^{a} | Treatment 2^{b} | Treatment 3^{c} | Dose (mg/kg) |
| 1 | 4 | Saline | Saline | Vehicle | 0 |
| 2 | 4 | Saline | Heparin | Cmpd. 100 | 8 |
| | | | (50 U/kg) | | |
| 3 | 4 | Saline | Heparin | Cmpd. 100 | 16 |
| | | | (50 U/kg) | | |
| 4 | 3 | DPH | Heparin | Cmpd. 100 | 8 |
| | | (10 mg/kg) | (50 U/kg) | | |
| 5 | 4 | DPH | Heparin | Cmpd. 100 | 16 |
| | | (10 mg/kg) | (50 U/kg) | | |
| 6 | 4 | CIM | Heparin | Cmpd. 100 | 8 |
| | | (20 mg/kg) | (50 U/kg) | | |
| 7 | 4 | CIM | Heparin | Cmpd. 100 | 16 |
| | | (20 mg/kg) | (50 U/kg) | | |
| 8 | 4 | L-NAME | Heparin | Cmpd. 100 | 8 |
| | | (100 mg/kg) | (50 U/kg) | | |
| 9 | 4 | L-NAME | Heparin | Cmpd. 100 | 16 |
| | | (100 mg/kg) | (50 U/kg) | | |
| 10 | 3 | DPH + CIM | Heparin | Cmpd. 100 | 8 |
| | | (10 mg/kg + | (50 U/kg) | | |
| | | 20 mg/kg) | | | |
| 11 | 4 | DPH + CIM | Heparin | Cmpd. 100 | 16 |
| | | (10 mg/kg + | (50 U/kg) | | |
| | | 20 mg/kg) | | | |

^{a}Administered at T = −15 minutes by i.v. bolus with the exception of DPH which was administered by subcutaneous injection
^{b}Administered at T = −3 minutes by i.v. bolus
^{c}Administered at T = 0 minutes by i.v. infusion over 10 minutes

The infusion of vehicle (Group 1) did not produce any statistically significant changes in blood pressure or heart rate when compared to the post-heparin values. Pretreatments with saline and unfractionated heparin at 50 U/kg IV did not significantly affect arterial blood pressure or heart rate. Treatment with Compound 100 at 8 and 16 mg/kg by IV infusion produced dose-dependent reductions in systolic, diastolic and mean arterial blood pressures at 1 minute following dosing when compared to the post-heparin values. The changes were transient and blood pressures returned to normal ranges at the later time points.

In rats pretreated with the H1 blocker diphenhydramine, the administration of Compound 100 at 8 or 16 mg/kg did not produce any significant changes in systolic, diastolic or mean arterial pressures at 1, 15 or 25 minutes following dosing. There were some small statistically-significant changes in blood pressures at 40 and 70 minutes following dosing but these changes were relatively small (<15%) and not considered biologically-relevant.

In rats pretreated with the H2 blocker cimetidine, the administration of Compound 100 at 8 mg/kg produced no significant change in systolic, diastolic or mean arterial pressures at any time point. However, administration of Compound 100 at 16 mg/kg produced statistically or biologically-significant reductions in blood pressures between 1 to 40 minutes following dosing.

In rats pretreated with the NO synthase inhibitor L-NAME, administration of Compound 100 at 8 and 16 mg/kg produced dose-dependent, statistically or biologically-significant reductions in blood pressures at 1 to 25 minutes following dosing. Blood pressures returned to normal ranges at 40 and 70 minutes post-treatment. Consistent with NO inhibition, increases in blood pressure were evident following administration of L-NAME, prior to dosing of test agent.

In rats pretreated with both the H1 and H2 blockers (diphenhydramine and cimetidine), administration of Compound 100 at 8 or 16 mg/kg did not produce any significant changes in blood pressures at any time point.

Statistically-significant reductions in heart rate were observed in many of the Compound 100-treated groups at 25 to 70 minutes post-treatment and were similar across the various treatment groups (Groups 2 to 11). However, the reductions were not dose-dependent and were considered a marginal effect over the heart rates observed in the vehicle-treated animals (Group 1).

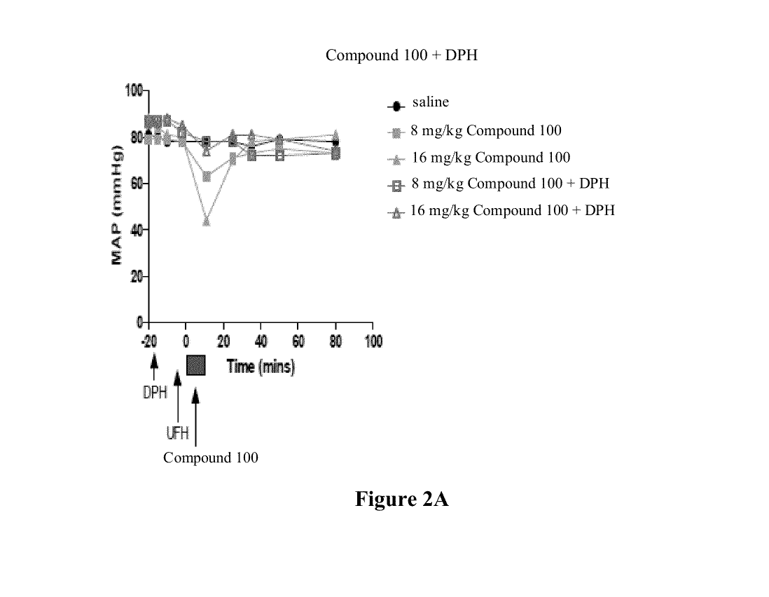

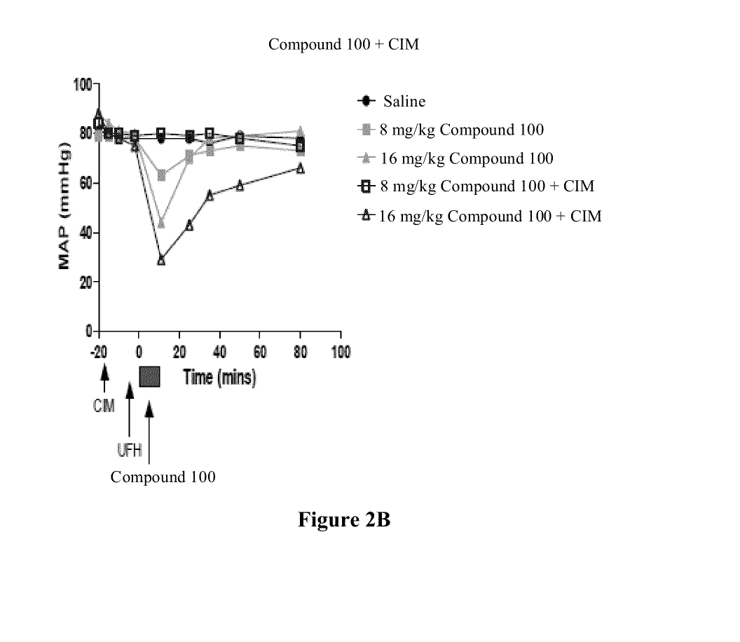

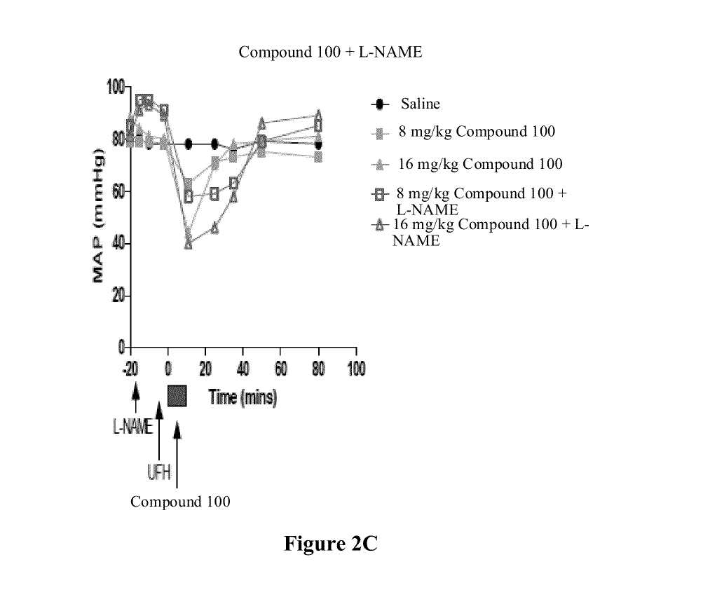

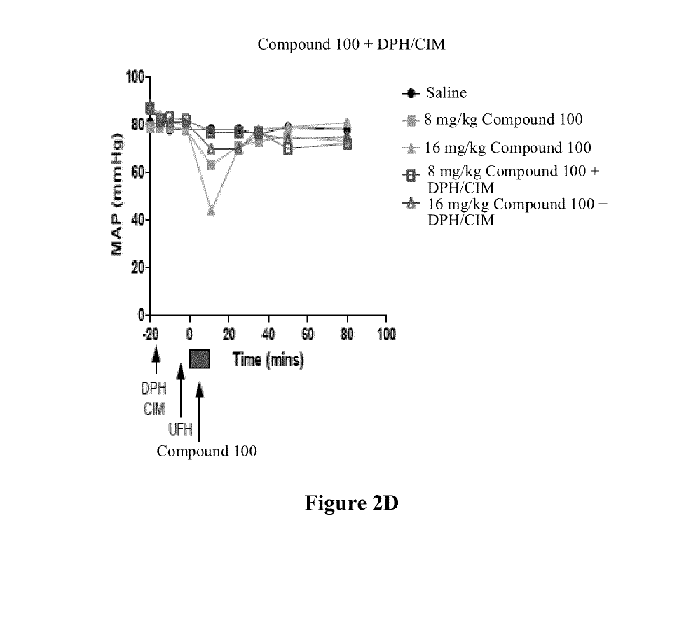

These results demonstrate that diphenhydramine effectively blocks blood pressure reductions caused by IV infusion of Compound 100 in the anesthetized rat. Cimetidine only prevented blood pressure reductions at the 8 mg/kg dose of Compound 100 but not the 16 mg/kg dosage. L-NAME, an NO synthase inhibitor, was ineffective at both Compound 100 dosages. This indicates that blood pressure reductions associated with Compound 100 administration in the anesthetized rat are largely caused by activation of the H1 receptor, presumably following histamine release. Results are shown in FIGS. 2A-2D.

Various modifications of the disclosure, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A pharmaceutical composition comprising:
a) one or more salicylamide compounds of Formula I: 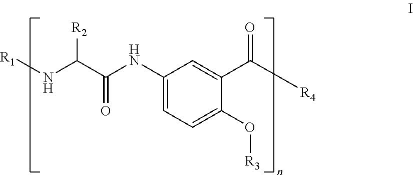
wherein:
n is 2 to 10;
R₁ is H or 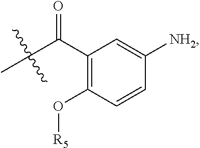
where R₅ is H or C₁ to C₉ straight or branched alkyl optionally substituted with one or more —NH₂, —N(CH₃)₂, or 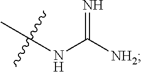 [SMILES: *NC(=N)N]
each R₂ is, independently, C₁ to C₉ straight or branched alkyl optionally substituted with one or more —NH₂, —N(CH₃)₂, or 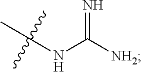 [SMILES: *NC(=N)N]
each R₃ is, independently, C₁ to C₉ straight or branched alkyl optionally substituted with one or more —NH₂, —N(CH₃)₂, or 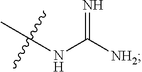 [SMILES: *NC(=N)N]
and
R₄ is OH, NH₂, or 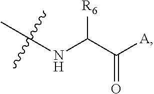
where A is OH or NH₂, and R₆ is H or C₁ to C₉ straight or branched alkyl optionally substituted with one or more —NH₂, —N(CH₃)₂, or 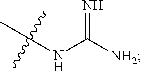 [SMILES: *NC(=N)N]
or a pharmaceutically acceptable salt thereof; and
b) one or more histamine blocking agents, or a pharmaceutically acceptable salt thereof.

2. The pharmaceutical composition of claim 1 wherein n is 3 to 8.

3. The pharmaceutical composition of claim 1 wherein n is 3 to 5.

4. The pharmaceutical composition of claim 1 wherein n is 3 or 4.

5. The pharmaceutical composition of claim 1 wherein R₁ is H.

6. The pharmaceutical composition of claim 1 wherein each R₂ is, independently, C₃ to C₅ straight or branched alkyl optionally substituted with one or more —NH₂ or

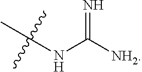 [SMILES: *NC(=N)N]

7. The pharmaceutical composition of claim 1 wherein each R₂ is, independently, C₃ or C₄ straight alkyl optionally substituted with one —NH₂ or

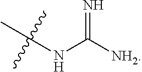 [SMILES: *NC(=N)N]

8. The pharmaceutical composition of claim 1 wherein each R₂ is, independently, C₃ or C₄ straight alkyl substituted with one —NH₂ or

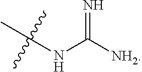 [SMILES: *NC(=N)N]

9. The pharmaceutical composition of claim 1 wherein each R₃ is, independently, C₁ to C₉ straight or branched alkyl.

10. The pharmaceutical composition of claim 1 wherein each R₃ is, independently, C₁ to C₃ straight alkyl.

11. The pharmaceutical composition of claim 1 wherein R₄ is OH, NH₂, or 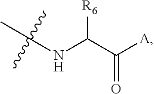
where A is NH₂, and R₆ is C₁ to C₉ straight or branched alkyl optionally substituted with one —NH₂, —N(CH₃)₂, or

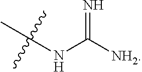 [SMILES: *NC(=N)N]

12. The pharmaceutical composition of claim 1 wherein R₄ is OH or NH₂.

13. The pharmaceutical composition of claim 1 wherein:
n is 3 to 5;
R₁ is H;
each R₂ is, independently, C₃ to C₅ straight alkyl optionally substituted with one —NH₂, —N(CH₃)₂, or 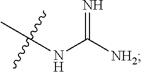 [SMILES: *NC(=N)N]
each R₃ is, independently, C₁ to C₃ straight alkyl optionally substituted with one —NH₂; and
R₄ is OH or NH₂.

14. The pharmaceutical composition of claim 1 wherein:
n is 3 or 4;
R₁ is H;
each R₂ is, independently, C₃ or C₄ straight alkyl substituted with one —NH₂ or 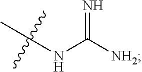 [SMILES: *NC(=N)N]
each R₃ is, independently, C₁ or C₂ alkyl; and
R₄ is NH₂.

15. The pharmaceutical composition of claim 1 wherein the salicylamide compound is chosen from: 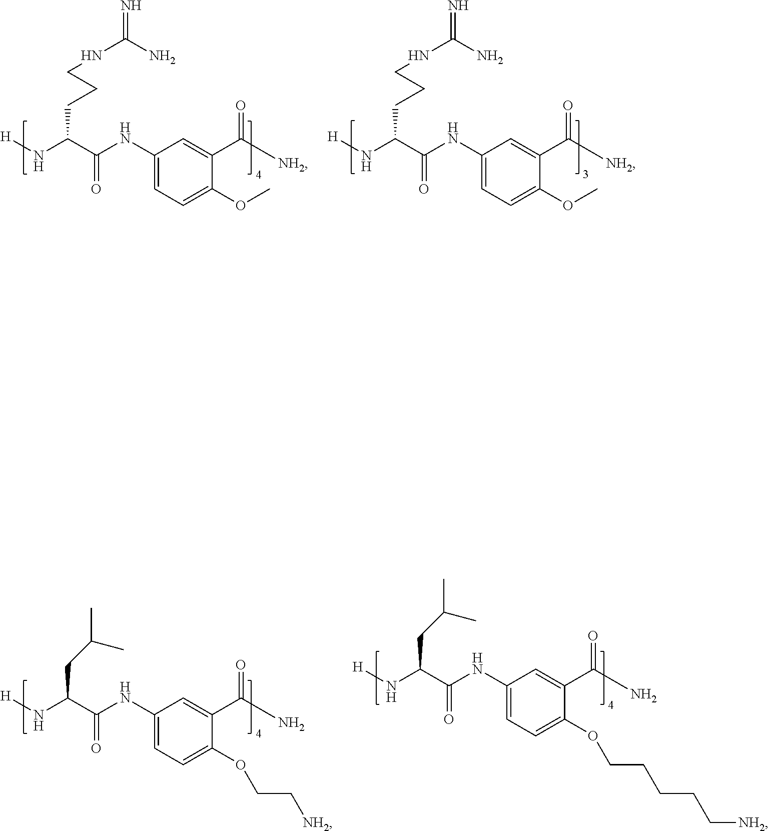 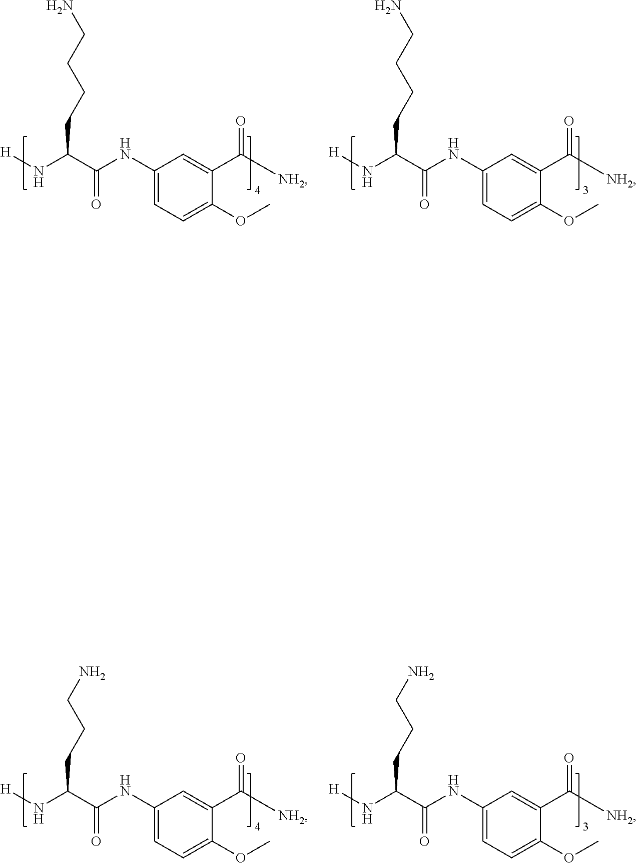 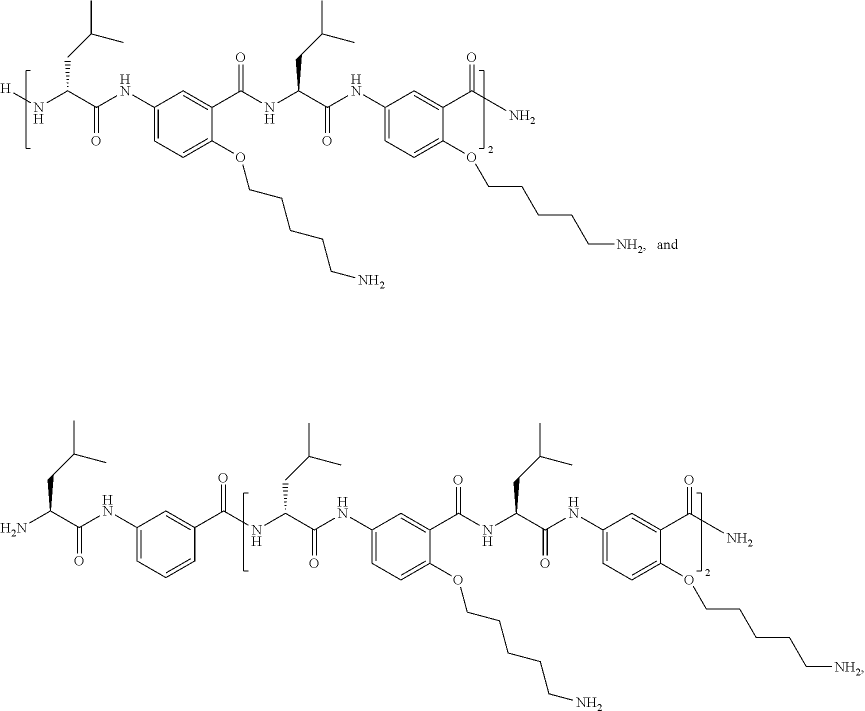
or a pharmaceutically acceptable salt thereof.

16. The pharmaceutical composition of claim 1 wherein the salicylamide compound is 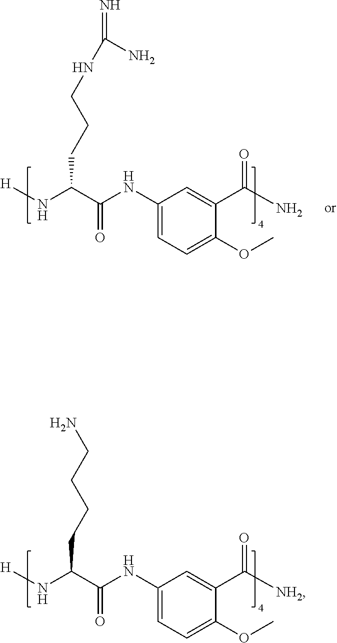
or a pharmaceutically acceptable salt thereof.

17. The pharmaceutical composition of claim 1 wherein the histamine blocking agent is chosen from diphenhydramine (Benadryl), cimetidine (Tagamet), loratadine (Claritin), fexofenadine (Allegra), chlorpheniramine (Chlor-Tripalon), brompheniramine (Dimetane), dimenhydrinate (Gravol), promethazine (Phenergan), hydroxyzine (Atarax), cyproheptadine (Periactin), azatadine (Zadine), and cetirizine (Reactine), or a pharmaceutically acceptable salt thereof, or any combination thereof.

18. The pharmaceutical composition of claim 1 wherein the histamine blocking agent is diphenhydramine or cimetidine, or a combination thereof.

19. The pharmaceutical composition of claim 1 wherein the salicylamide compound is present as a unit dose amount from about 5 mg to about 60 mg, and the histamine blocking agent is present as a unit dose amount from about 10 mg to about 50 mg.

20. The pharmaceutical composition of claim 1 wherein the salicylamide compound is present as a unit dose amount from about 10 mg to about 50 mg, and the histamine blocking agent is present as a unit dose amount from about 20 mg to about 40 mg.

21. The pharmaceutical composition of claim 1 wherein the salicylamide compound is 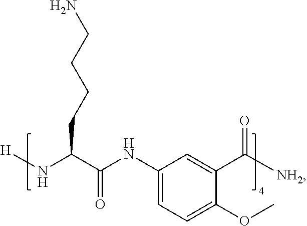
or a pharmaceutically acceptable salt thereof, and the histamine blocking agent is diphenhydramine or cimetideine, or a combination thereof.

22. A method of antagonizing unfractionated heparin, low molecular weight heparin, or a heparin/low molecular weight heparin derivative in a mammal comprising administering the composition of claim 1 to the mammal.

23. A method of antagonizing unfractionated heparin, low molecular weight heparin, or a heparin/low molecular weight heparin derivative in a mammal comprising:
administering one or more histamine blocking agents to the mammal; and
administering one or more salicylamide compounds to the mammal.

24-58. (canceled)
